(19) United States
(12) Patent Application Publication
(10) Pub. No.: US 2026/0364088 A1
(43) Pub. Date: Nov. 19, 2026

(54) TASL MIMICKING MOLECULES AND APPLICATIONS THEREOF

(71) Applicants: CEMM - FORSCHUNGSZENTRUM FÜR MOLEKULARE MEDIZIN GMBH, Vienna (AT); MEDICAL UNIVERSITY OF VIENNA, Vienna (AT)

(72) Inventors: Giulio SUPERTI-FURGA, Vienna (AT); Andras BOESZOERMENYI, Vienna (AT); Leonhard HEINZ, Vienna (AT); Manuele REBSAMEN, Vienna (AT)

(73) Assignees: CEMM - FORSCHUNGSZENTRUM FÜR MOLEKULARE MEDIZIN GMBH, Vienna (AT); MEDICAL UNIVERSITY OF VIENNA, Vienna (AT)

(21) Appl. No.: 19/123,752

(22) PCT Filed: Oct. 24, 2023
(86) PCT No.: PCT/EP2023/079674

§ 371 Date: Apr. 23, 2025

(30) Foreign Application Priority Data
Oct. 24, 2022 (EP) 22203423.3
Oct. 20, 2023 (EP) 23204997.3

(51) Int. Cl. A61K 31/55; A61K 31/4709; A61P 37/02; C07D 215/52; C07D 401/12

(52) U.S. Cl. CPC A61K 31/55; A61K 31/4709; A61P 37/02; C07D 215/52; C07D 401/12

(57) ABSTRACT

The present invention relates to a compound of formula (I) or its pharmaceutically acceptable salt. The present invention further relates to a pharmaceutical composition comprising the compound of formula (I) or its pharmaceutically acceptable salt, and at least one pharmaceutically acceptable carrier. The compounds of formula (I) are useful as medicaments, for use in treatment or prevention of an autoimmune disorder or inflammatory condition, in particular an autoimmune disorder being systemic lupus erythematosus, or an inflammatory condition selected from inflammatory bowel disease, psoriasiform dermatitis, and endosomal TLR-dependent inflammation. Preferably, the autoimmune disorder to be treated with the compound of formula (I) is a disorder associated with SLC15 peptide transporter. The compound of formula (I) has been demonstrated to inhibit SLC15 peptide transporter.

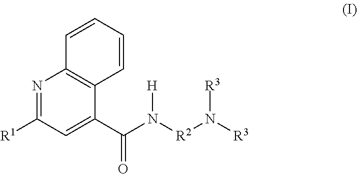

### FIELD OF THE INVENTION

The present invention relates to a compound of formula (I) or its pharmaceutically acceptable salt. The present invention further relates to a pharmaceutical composition comprising the compound of formula (I) or its pharmaceutically acceptable salt, and at least one pharmaceutically acceptable carrier. The compounds of formula (I) are useful as medicaments, for use in treatment or prevention of an autoimmune disorder or inflammatory condition, in particular an autoimmune disorder being systemic lupus erythematosus, or an inflammatory condition selected from inflammatory bowel disease, psoriasiform dermatitis, and endosomal TLR-dependent inflammation. Preferably, the autoimmune disorder to be treated with the compound of formula (I) is a disorder associated with SLC15 peptide transporter. The compound of formula (I) has been demonstrated to inhibit SLC15 peptide transporter.

### BACKGROUND OF THE INVENTION

Dysregulation of pathogen-recognition pathways of the innate immune system is associated with multiple autoimmune disorders. Due to the intricacies of the molecular network involved, the identification of pathway- and disease-specific therapeutics has been challenging.

Recognition of pathogen-derived nucleic acids by pattern recognition receptors is essential to mount protective innate immune responses (E. Bartok, G. Hartmann, Immune Sensing Mechanisms that Discriminate Self from Altered Self and Foreign Nucleic Acids. Immunity 53, 54-77 (2020); A. L. Blasius, B. Beutler, Intracellular toll-like receptors. Immunity 32, 305-315 (2010); A. Ablasser, Z. J. Chen, cGAS in action: Expanding roles in immunity and inflammation. Science 363, (2019); N. A. Lind, V. E. Rael, K. Pestal, B. Liu, G. M. Barton, Regulation of the nucleic acid-sensing Toll-like receptors. Nat Rev Immunol 22, 224-235 (2022); K. A. Fitzgerald, J. C. Kagan, Toll-like Receptors and the Control of Immunity. Cell 180, 1044-1066 (2020)). Despite tight regulatory mechanisms, aberrant activation of these pathways by endogenous ligands or mutations in key regulatory components lead to excessive responses that are causatively linked to several autoimmune conditions (K. Pelka, T. Shibata, K. Miyake, E. Latz, Nucleic acid-sensing TLRs and autoimmunity: novel insights from structural and cell biology. Immunol Rev 269, 60-75 (2016); Y. J. Crow, D. B. Stetson, The type I interferonopathies: 10 years on. Nat Rev Immunol 22, 471-483 (2022)). In particular, sensing of nucleic acids by endolysosomal TLRs is thought to be critically involved in the etiology of systemic lupus erythematosus and related autoimmune diseases as well as inflammatory conditions (G. J. Brown et al., TLR7 gain-of-function genetic variation causes human lupus. Nature 605, 349-356 (2022); S. Fillatreau, B. Manfroi, T. Dorner, Toll-like receptor signalling in B cells during systemic lupus erythematosus. Nat Rev Rheumatol 17, 98-108 (2021); G. C. Tsokos, M. S. Lo, P. Costa Reis, K. E. Sullivan, New insights into the immunopathogenesis of systemic lupus erythematosus. Nat Rev Rheumatol 12, 716-730 (2016)). Both human genetic and mouse studies have unequivocally identified the lysosomal solute carrier SLC15A4 and transcription factor IRF5 as components essential for mediating disease development downstream of TLRs (T. Ban, G. R. Sato, T. Tamura, Regulation and role of the transcription factor IRF5 in innate immune responses and systemic lupus erythematosus. Int Immunol 30, 529-536 (2018); J. Bentham et al., Genetic association analyses implicate aberrant regulation of innate and adaptive immunity genes in the pathogenesis of systemic lupus erythematosus. Nat Genet 47, 1457-1464 (2015); A. L. Blasius et al., Slc15a4, AP-3, and Hermansky-Pudlak syndrome proteins are required for Toll-like receptor signaling in plasmacytoid dendritic cells. Proc Natl Acad Sci USA 107, 19973-19978 (2010); T. Kobayashi et al., The histidine transporter SLC15A4 coordinates mTOR-dependent inflammatory responses and pathogenic antibody production. Immunity 41, 375-388 (2014); R. R. Graham et al., A common haplotype of interferon regulatory factor 5 (IRF5) regulates splicing and expression and is associated with increased risk of systemic lupus erythematosus. Nat Genet 38, 550-555 (2006); A. Katewa et al., The peptide symporter SLC15a4 is essential for the development of systemic lupus erythematosus in murine models. PLOS One 16, e0244439 (2021); H. Almuttaqi, I. A. Udalova, Advances and challenges in targeting IRF5, a key regulator of inflammation. FEBS J 286, 1624-1637 (2019); J. W. Han et al., Genome-wide association study in a Chinese Han population identifies nine new susceptibility loci for systemic lupus erythematosus. Nat Genet 41, 1234-1237 (2009); S. Song et al., Inhibition of IRF5 hyperactivation protects from lupus onset and severity. J Clin Invest 130, 6700-6717 (2020); T. Ban et al., Genetic and chemical inhibition of IRF5 suppresses pre-existing mouse lupus-like disease. Nat Commun 12, 4379 (2021)). Investigating the mechanistic involvement of SLC15A4, we recently discovered the protein TASL, encoded by an SLE-associated gene previously known as CXorf21, as an interactor essential for IRF5 activation (Bentham et al., 2015; L. X. Heinz et al., TASL is the SLC15A4-associated adaptor for IRF5 activation by TLR7-9. Nature 581, 316-322 (2020); C. A. Odhams et al., Interferon inducible X-linked gene CXorf21 may contribute to sexual dimorphism in Systemic Lupus Erythematosus. Nat Commun 10, 2164 (2019)). Through a C-terminal pLxIS motif, TASL acts as signaling adaptor mediating recruitment of IRF5, in analogy to the key immune adaptors MAVS, STING and TRIF for IRF3 (Heinz et al., 2020; S. Liu et al., Phosphorylation of innate immune adaptor proteins MAVS, STING, and TRIF induces IRF3 activation. Science 347, aaa2630 (2015)). Thus, TASL represents the fourth, central element in a pathway in which each component is associated with SLE, providing an unusually strong case and rationale for therapeutic intervention. Interfering with SLC15A4-TASL complex formation has been shown to abolish TLR-induced IRF5 activation, suggesting that this pathway can be targeted with high specificity.

Here we describe the identification of a chemical entity, dTASL1, which inhibits signaling of the nucleic acid-sensing TLR7/8 pathway leading to IRF5 activation. dTASL1 interferes with assembly of the SLC15A4-TASL module, leading to efficient degradation of TASL and ablation of IRF5 activation. Consequently, dTASL1 blocks endolysosomal TLR-induced responses in disease-relevant human immune cells.

WO 2014/034719 discloses certain compounds capable of inhibiting TLR3, 7 and/or 9, which have an excellent prophylactic and/or therapeutic effect on autoimmune diseases, inflammation, allergies, asthma, graft rejection, GvHD or cardiomyopathy associated with sepsis.

### SUMMARY OF THE INVENTION

There is an urgent need for new treatments and therapeutic modalities against autoimmune diseases, in particular against systemic lupus erythematosus, as well as inflammation conditions. The present invention addresses this problem through provision of new chemical entities useful in the treatment or prevention of autoimmune diseases, in particular against systemic lupus erythematosus, as well as inflammation conditions.

The present inventors have demonstrated that chemical targeting of an endolysosomal signaling complex inhibits an SLE-associated proinflammatory pathway. The present inventors have found that chemical intervention with the compound of formula (I) leads to efficient catalytic degradation of TASL protein. Without being bound by the theory, the present inventors have demonstrated that, TASL is regulated by proteostatic interaction with SLC15A4. Accordingly, interfering with complex formation alone has been shown to lead to efficient catalytic degradation of TASL protein. Accordingly, the present invention is based at least in part on the discovery that exploiting this property may be advantageous for chemical targeting of TASL.

Accordingly, the present invention provides a highly specific degrader of TASL which specifically interferes with IRF5 signalling. Considering the specificity of dTASL1 in targeting IRF5 signaling and that all components involved have been genetically associated with systemic lupus erythematosus (SLE), the study represents a proof-of-concept for the development of new therapeutics against this disease.

The invention will be summarized in the following embodiments.

In a first embodiment, the present invention relates to a compound of formula (I):

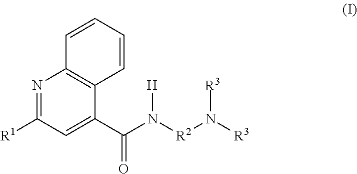

or a pharmaceutically acceptable salt thereof, wherein:
R¹ is aryl or heteroaryl, optionally substituted with one or more R^{S2},
R² is C₂₋₅ alkylene, optionally substituted with one or more R^{S1},
each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said (C₀₋₃ alkylene)-aryl and the heteroaryl moiety in said-(C₀₋₃ alkylene)-heteroaryl are each optionally substituted with one or more R^{S2}; or
both R³ are joined together to form together with the N atom that otherwise carries both R³ a heterocyclic ring, optionally substituted with one or more R^{S2}; or —R²—NR³R³ are taken together to form:

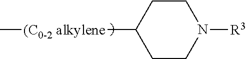

wherein R³ is selected from C₁₋₅ alkyl, C₂₋₅ alkenyl, C₂₋₅ alkynyl, (C₀₋₃ alkylene)-cycloalkyl, (C₀₋₃ alkylene)-heterocycloalkyl, (C₀₋₃ alkylene)-aryl and (C₀₋₃ alkylene)-heteroaryl, wherein said alkyl, said alkenyl, said alkynyl and the alkylene moiety in said —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2};
wherein each R^{S1} is independently selected from —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl); and wherein each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), —SO—(C₁₋₆ alkyl), —(C₀₋₄ alkylene)-carbocyclyl, and —(C₀₋₄ alkylene)-heterocyclyl, wherein the carbocyclyl group in said-(C₀₋₄ alkylene)-carbocyclyl and the heterocyclyl group in said-(C₀₋₄ alkylene)-heterocyclyl are each optionally substituted with one or more groups independently selected from C₁₋₆ alkyl, C₂₋₆ alkenyl, C₂₋₆ alkynyl, halogen, C₁₋₆ haloalkyl, —O—(C₁₋₆ haloalkyl), —CN, —OH, —O(C₁₋₆ alkyl), —SH, —S(C₁₋₆ alkyl), —NH₂, —NH(C₁₋₆ alkyl), —N(C₁₋₆ alkyl) (C₁₋₆ alkyl), —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).

In a second embodiment, the present invention relates to a pharmaceutical composition comprising the compound of formula (I) or a pharmaceutically acceptable salt thereof, and at least one pharmaceutically acceptable carrier.

In a third embodiment, the present invention relates to the compound of formula (I) or a pharmaceutically acceptable salt thereof, or to the pharmaceutical composition of the present invention for use as a medicament.

In a fourth embodiment, the present invention relates to the compound of formula (I) or a pharmaceutically acceptable salt thereof, or to the pharmaceutical composition of the present invention for use in treatment or prevention of an autoimmune disorder or inflammatory condition.

In a fifth embodiment, the present invention relates to use of the compound of formula (I) or a pharmaceutically acceptable salt thereof, or the pharmaceutical composition of the present invention in the manufacture of a medicament for treatment or prevention of an autoimmune disorder or inflammatory condition.

In a sixth embodiment, the present invention relates to the method of treatment of an autoimmune disorder or inflammatory condition, the method comprising administering the compound of formula (I) or a pharmaceutically acceptable salt thereof, or the pharmaceutical composition of the present invention, to the subject in need thereof. It is to be understood that a therapeutically effective amount of the compound of formula (I) or its salt, or the pharmaceutical composition of the present invention, is to be administered.

### BRIEF DESCRIPTION OF FIGURES

The invention is further illustrated by the following Figures. The figures serve illustrative purpose and shall not be construed as limiting the invention in any way.

FIG. 1 shows SLC15A4-TASL binding interface controlling TASL stability (B) AlphaFold model of SLC15A4 in complex with amino terminus of TASL.

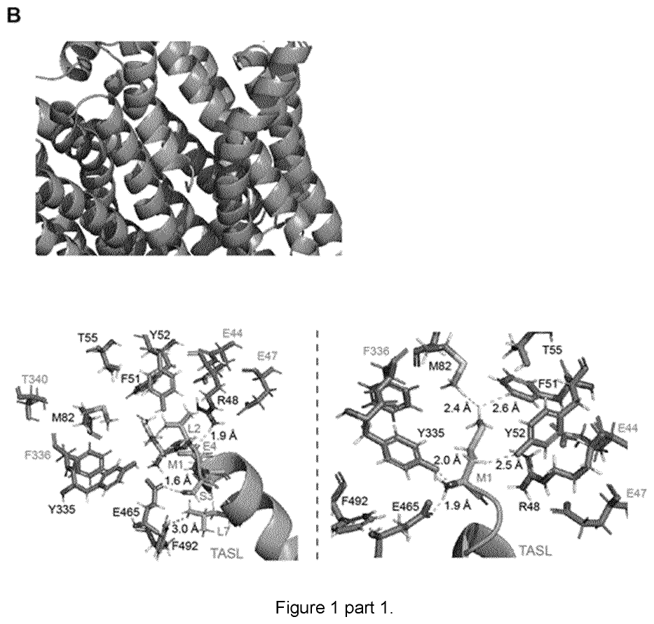

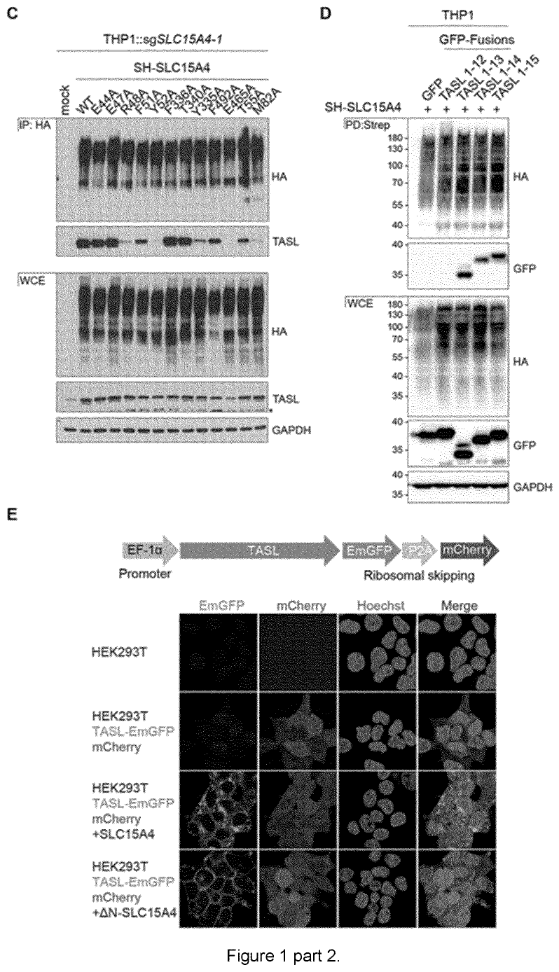

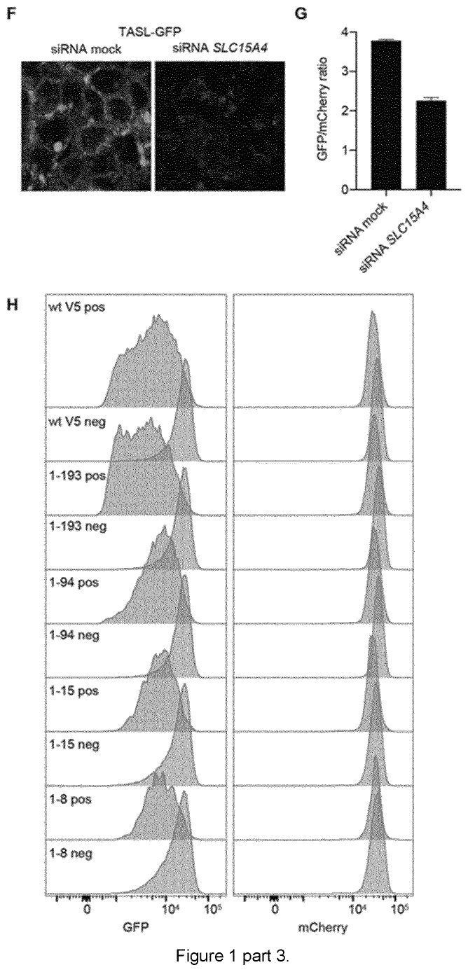

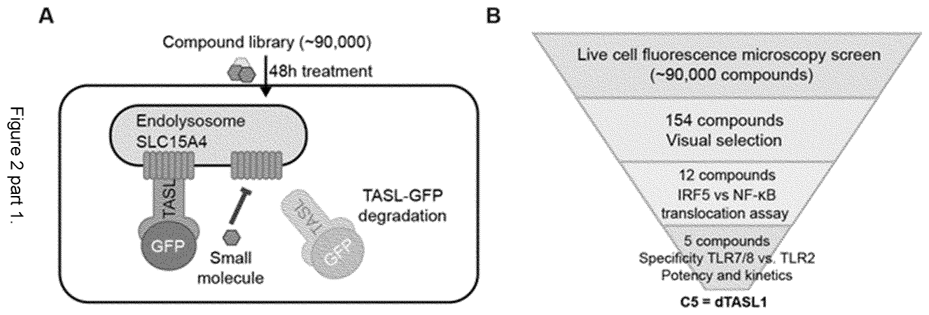

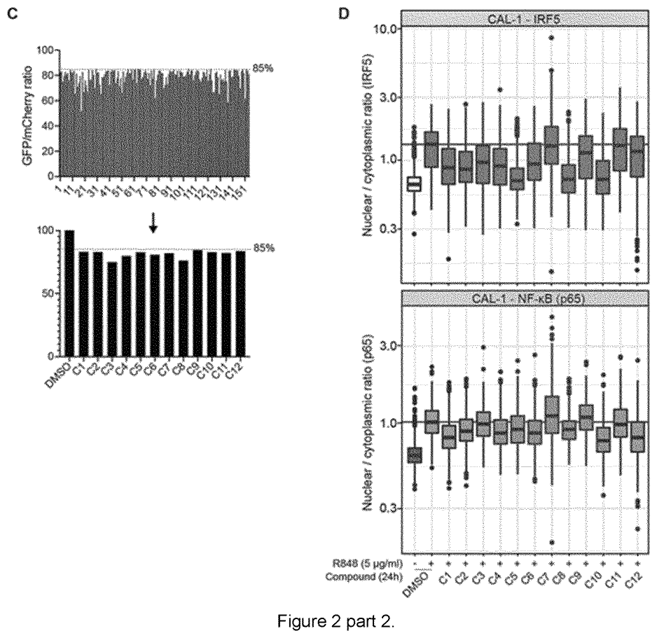

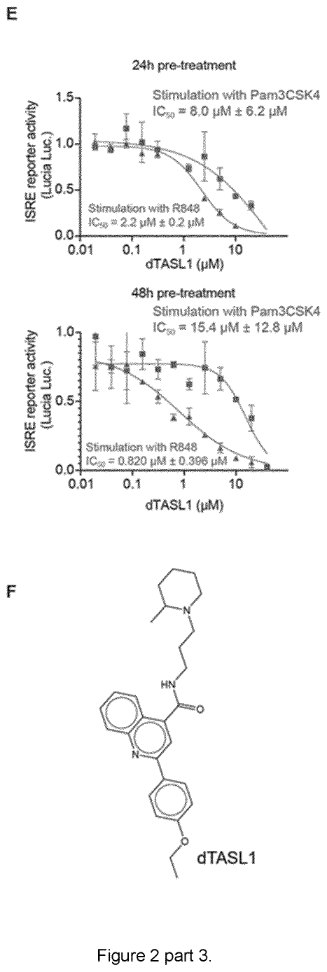

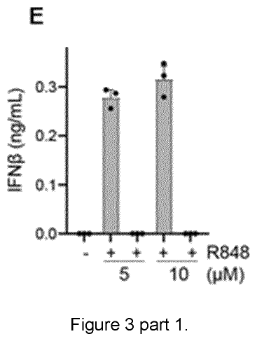

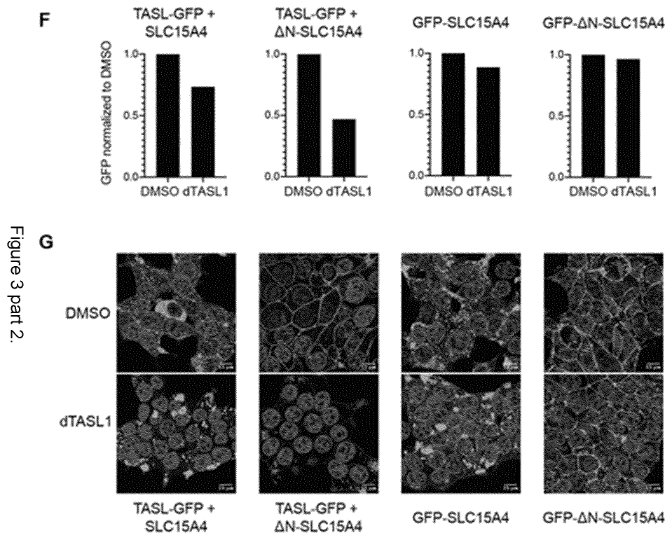

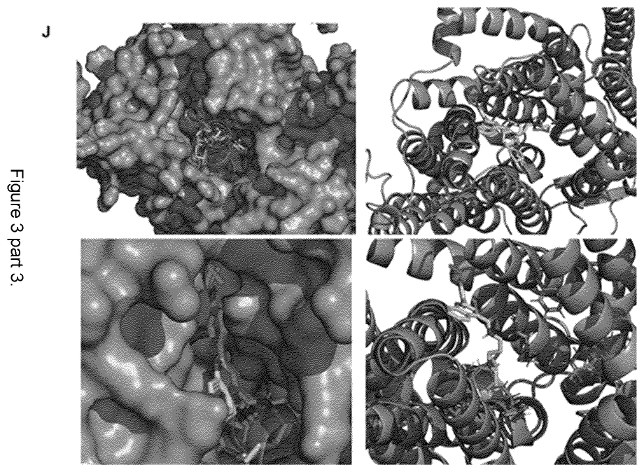

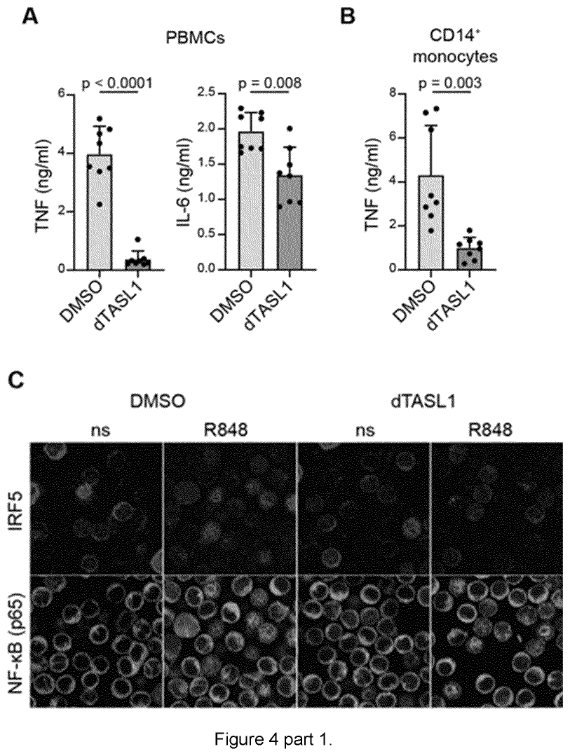

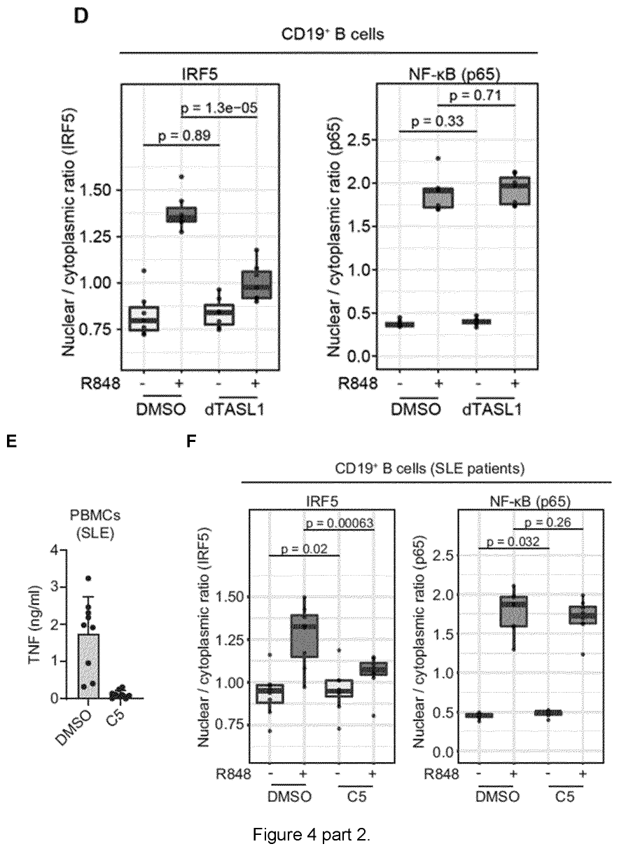

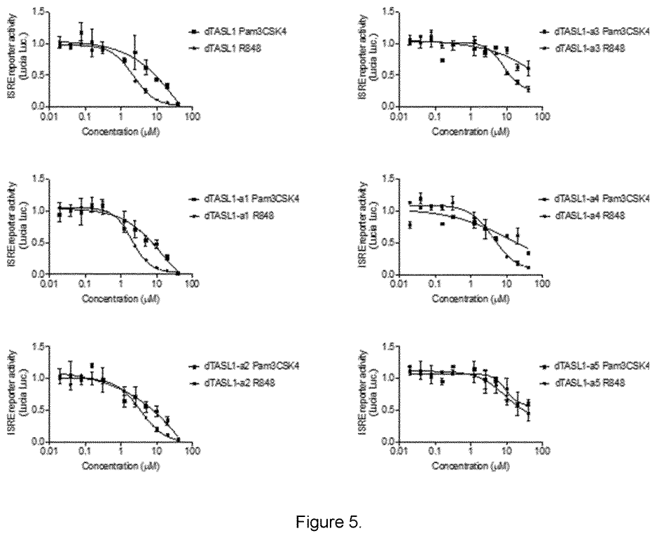

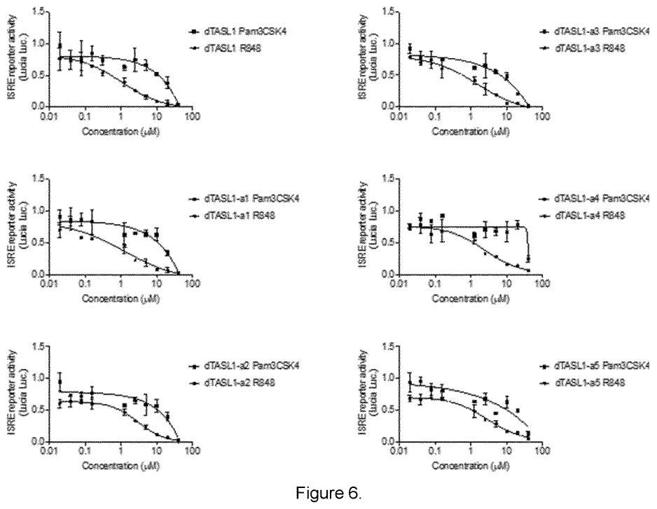

Residues determined to be critical for the interaction by co-immunoprecipitation in FIG. 1C as well as adjacent residues that have no effect on the interaction are shown. (C) Immunoblots show WCE and HA immunoprecipitates from sgSLC15A4-expressing THP1 cells reconstituted with the indicated StepHA-tagged wildtype (WT) or mutant SLC15A4 variants. (D) Immunoblots show WCE and StrepTactin pull-downs (PD) from StrepHA-SLC15A4-expressing THP1 cells co-expressing the indicated GFP-fusion constructs. (E) Schematic of the TGC vector (top) and confocal live cell microscopy images of HEK293T stably expressing the TGC vector, SLC15A4 and/or its plasma membrane localized variant ΔN-SLC15A4 as indicated (bottom). (F, G) Live cell confocal microscopy images (F) and GFP/mCherry fluorescence ratios (G) of the TGC reporter clone 48 h after transfection with the indicated siRNA. (H) Flow-cytometry analysis of TGC reporter clone transfected with doxycycline-inducible V5-tagged TASL fragments and stained 24 h after induction with anti-V5 antibodies. GFP or mCherry fluorescence intensities were assessed in V5 positive and negative cells. In (D-H) data are representative of at least two independent experiments.

FIG. 2 shows chemical screen for TASL-destabilizing molecules identifies dTASL1 as IRF5 pathway-specific compound. (A) Schematic of screening principle to identify TASL-destabilizing compounds. (B) Summary of compound evaluation steps and validation assays leading to dTASL1 selection. (C) POC GFP/mCherry ratios of 154 compounds identified by the image analysis pipeline (top) and of 12 compounds selected after visual inspection (bottom). (D) Image-based evaluation of IRF5 (top) and NF-kB (bottom) nuclear translocation in CAL-1 cells. Cells were pre-treated for 24 h with the indicated compounds (10 μM) or DMSO, stimulated with R848 (5 μg/ml) for 3 h as indicated and analyzed by confocal microscopy. Box plots show cellular nuclear to cytoplasmic intensity ratios. (E) Dose response of dTASL1 in THP1 ISRE reporter cells. Cells were pre-treated for 24 h (top) and 48 h (bottom) before stimulation with R848 (5 μg/ml) or Pam3CSK4 (0.1 μg/ml). (G) Chemical structure of dTASL1. In (D, E) data are representative of at least two independent experiments.

FIG. 3 shows dTASL1 acts through SLC15A4 to selectively inhibit TLR7/8-induced IRF5 activation and pro-inflammatory responses. (E) Supernatant from CAL-1 (E) cells pre-treated with dTASL1 for 24 h before R848 (5 μg/ml, 16-24 h) stimulation were analysed by ELISA for the indicated cytokines. (F, G) Flowcytometry analysis (F) or live cell fluorescence microscopy images (G) of HEK293T cells stably expressing the indicated constructs after 24 h treatment with DMSO or 10 UM dTASL1. In (F) integrated GFP signal is shown. (J) Molecular docking of dTASL1 onto SLC15A4 AlphaFold model. (Left) The four lowest energy structures of dTASL1 are shown with an overlay of the N-terminal TASL helix (green).

The aromatic 2-(4-ethoxyphenyl) quinoline moiety of dTASL1 is placed in a cavity above the TASL binding channel. The 2-methylpiperidine moiety is predicted to hover above the peptide-binding channel (depicted by the representation of four lowest energy binding modes). The steric clash with TASL is visualized with an overlay showing the N-terminal TASL helix and dTASL1 present in the same position. (Right) The four lowest energy structures of dTASL1 are shown above residues required for TASL binding along with adjacent residues that are not required for binding (related to FIG. 1B-C) (top). The lowest energy dTASL1 binding mode is depicted (bottom). In (E-I) data are representative of at least two independent experiments.

FIG. 4 shows dTASL1 selectively impairs IRF5-dependent activation in primary human cells. (A) PBMCs or (B) isolated CD14⁺ monocytes from healthy donors were pre-treated for 24 h with dTASL1 (10 UM) and stimulated for 16 h with R848 (5 μg/ml). Cellular supernatants were analyzed by ELISA as indicated. Data show mean±SD, n=8. Significance was assessed by paired t test. (C) Representative confocal microscopy images of PBMCs pre-treated for 24 h with dTASL1 (10 UM) and stimulated for 3 h with R848 (5 μg/ml) stained for IRF5 and NF-κB p65. (D) PBMCs of 7 individual donors pre-treated for 24 h with DMSO or dTASL1 (10 UM) and stimulated with R848 (5 μg/ml) for 3 h as indicated were analyzed for IRF5 and NF-κB p65 nuclear translocation. Box plots show CD19⁺ B cell-specific median cellular nuclear to cytoplasmic intensity ratio per individual donor. n=7. (E) PBMCs from SLE patients were pretreated for 24 h with dTASL1 (also referred to as C₅) (10 UM) and stimulated for 16 h with R848 (5 μg/ml). Supernatants were analyzed by ELISA as indicated. Data show mean±SD, n=9 donors. (F) PBMCs of 8 SLE patients pre-treated for 24 h with DMSO or dTASL1 (also referred to as C5) (10 UM) and stimulated with R848 (5 μg/ml) for 3 h as indicated were analyzed for CD19⁺ B cell-specific IRF5 and NF-κB p65 nuclear translocation. n=8 donors. Significance was assessed by paired t test.

FIG. 5 shows dose response curves with dTASL1 and analogues after 24 h compound treatment followed by 16 h R848 or Pam3CSK4 stimulation, respectively. Data are representative of two independent experiments.

FIG. 6 shows dose response curves with dTASL1 and analogues after 48 h compound treatment followed by 16 h R848 or Pam3CSK4 stimulation, respectively. Data are representative of three independent experiments.

### DEFINITIONS

The following definitions apply throughout the present specification and the claims, unless specifically indicated otherwise.

The term “hydrogen” is herein used to refer to protium, deuterium and/or tritium, preferably to protium. Accordingly, the term “non-hydrogen atom” refers to any atoms that is not hydrogen, i.e. that is not protium, deuterium or tritium.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂-or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl. Preferably, the term heterocyclic ring refers to heteroaryl ring, heterocycloalkyl ring or heterocycloalkenyl ring, more preferably the term heterocyclic ring refers to heterocycloalkyl ring.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., ¹H indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members (e.g., cyclopropyl or cyclohexyl).

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “halogen” or “HaI” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is-CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to +10% of the indicated numerical value, more preferably to +5% of the indicated numerical value, and in particular to the exact numerical value indicated. If the term “about” is used in connection with the endpoints of a range, it preferably refers to the range from the lower endpoint-10% of its indicated numerical value to the upper endpoint +10% of its indicated numerical value, more preferably to the range from of the lower endpoint-5% to the upper endpoint +5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician (or attendant veterinarian).

### DETAILED DESCRIPTION OF THE INVENTION

The invention will be described in detail in the following.

In one embodiment, the present invention relates to a compound of formula (I):

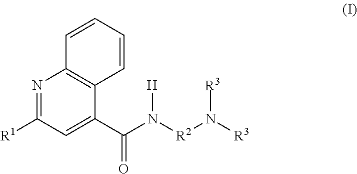

or a pharmaceutically acceptable salt thereof.

In formula (I), R¹ is aryl or heteroaryl, optionally substituted with one or more R^{S2}.

Preferably, R¹ is aryl, optionally substituted with one or more R^{S2}. Particularly suitable aryl moiety is phenyl. Accordingly and preferably, R¹ is phenyl, optionally substituted with one or more R^{S2}. More preferably, R¹ is phenyl optionally substituted with —O(C₁₋₆ alkyl). Suitable substituents include methoxy, ethoxy, n-propoxy, isopropoxy and n-butoxy groups. Particularly preferred is ethoxy group. Accordingly and preferably R¹ is selected from 4-methoxyphenyl, 4-ethoxyphenyl, 4-n-propoxyphenyl, 4-isopropoxyphenyl and 4-n-butoxyphenyl, more preferably R¹ is 4-ethoxyphenyl.

In certain preferred embodiments of the invention, it is preferred that R¹ is substituted with one or more R^{S2}. Thus, it is preferred that R¹ is aryl or heteroaryl, wherein the aryl or heteroaryl is substituted with one or more R^{S2}.

Preferably, R¹ is aryl, which is substituted with one or more R^{S2}. Particularly suitable aryl moiety is phenyl, which is substituted with one or more R^{S2}. Accordingly and preferably, R¹ is phenyl, which is substituted with one or more R^{S2}. As indicated above, R^{S2} is preferably-O(C₁₋₆ alkyl), more preferably ethoxy.

In formula (I), R² is C₂₋₅ alkylene, optionally substituted with one or more R^{S1}. It is preferred that the distance between the two attachments points in said C₂₋₅ alkylene in R² is three carbon atoms, and accordingly C₂₋₅ alkylene in R² is preferably selected from —CH₂CH₂CH₂—, —CH(CH₃)CH₂CH₂—, —CH₂CH(CH₃)CH₂—, and —CH₂CH₂CH(CH₃)—, wherein said alkylene group is optionally substituted with one or more R^{S1}. It is to be understood that preferably the attachment point presented on the left side of the formula representing the alkylene moiety in R² is connected to the nitrogen atom of —C(O)NH-moiety, and the attachment point presented on the right side of the formula representing the alkylene moiety in R² is connected to the nitrogen atom of —NR³R³ moiety. More preferably, R² is selected from —CH₂CH₂CH₂—, and —CH₂CH(CH₃)CH₂—.

In certain preferred embodiments, R² is C₂₋₄ alkylene. Preferably R² is selected from —CH₂CH₂—, —CH₂CH₂CH₂—, —CH₂CH₂CH₂CH₂—, and —CH₂CH(CH₃)CH₂—. Particularly preferably R² is-CH₂CH₂CH₂—.

In other preferred embodiments, R² is C₃₋₄ alkylene. Preferably R² is selected from —CH₂CH₂CH₂—, —CH₂CH₂CH₂CH₂—, and —CH₂CH(CH₃)CH₂—. Particularly preferably R² is-CH₂CH₂CH₂—.

In formula (I), each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said —(C₀₋₃ alkylene)-aryl and the heteroaryl moiety in said-(C₀₋₃ alkylene)-heteroaryl are each optionally substituted with one or more R^{S2}; or
both R³ are joined together to form together with the N atom that otherwise carries both R³ a heterocyclic ring, optionally substituted with one or more R^{S2}; or —R²—NR³R³ are taken together to form:

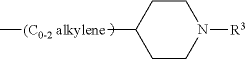

wherein R³ is selected from C₁₋₅ alkyl, C₂₋₅ alkenyl, C₂₋₅ alkynyl, (C₀₋₃ alkylene)-cycloalkyl, (C₀₋₃ alkylene)-heterocycloalkyl, (C₀₋₃ alkylene)-aryl and (C₀₋₃ alkylene)-heteroaryl, wherein said alkyl, said alkenyl, said alkynyl and the alkylene moiety in said —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2}.

Preferably, each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said —(C₀₋₃ alkylene)-aryl and the heteroaryl moiety in said-(C₀₋₃ alkylene)-heteroaryl are each optionally substituted with one or more R^{S2}; or
both R³ are joined together to form together with the N atom that otherwise carries both R³ a heterocyclic ring, optionally substituted with one or more R^{S2}.

In certain preferred embodiments, each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said-(C₀₋₃ alkylene)-aryl are each optionally substituted with one or more R^{S2}. Preferably, each R³ is independently selected from methyl, ethyl, propyl, isopropyl, butyl, isobutyl, cyclohexyl and benzyl. It is further preferred that the proviso applies that not both R³ are unsubstituted methyl and that the proviso applies that not both R³ are unsubstituted ethyl.

In other preferred embodiments, both R³ are joined together to form together with the N atom that otherwise carries both R³ a heterocyclic ring optionally substituted with one or more R^{S2}. Preferably, the heterocyclic ring is a heterocycloalkyl ring. Thus, it is preferred that both R³ are joined together to form together with the N atom that otherwise carries both R³ a heterocycloalkyl ring optionally substituted with one or more R^{S2}.

More preferably, both R³ are joined together to form together with the N atom that otherwise carries both R³ a heterocyclic ring, optionally substituted with one or more R^{S2}. Preferably, the heterocyclic ring optionally substituted with one or more R^{S2} is a heterocyclic ring optionally substituted with one or more C₁₋₆ alkyl groups. Preferably, the heterocyclic ring is selected from piperidine, pyrrolidine and azepane, more preferably the heterocyclic ring is selected from piperidine and pyrrolidine, even more preferably the heterocyclic ring is piperidine. Accordingly and preferably, the —NR³R³ is selected from:

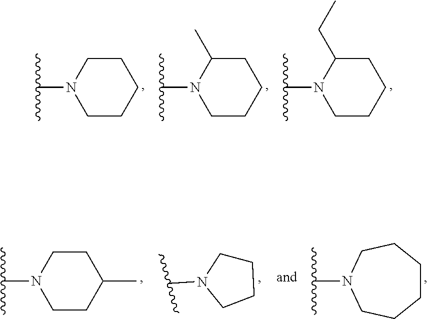

preferably selected from

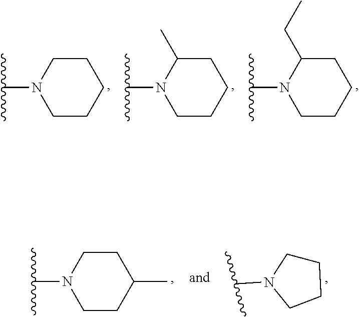

more preferably selected from

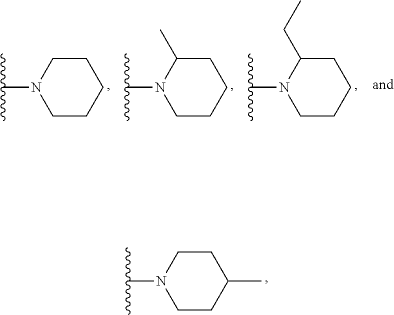

even more preferably the —NR³R³ is

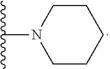 [SMILES: *N1CCCCC1]

In other preferred embodiments, —R²—NR³R³ are taken together to form:

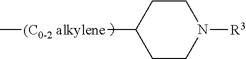

preferably wherein R³ is selected from methyl, ethyl, propyl, isopropyl, butyl, isobutyl, cyclohexyl and benzyl.

In formula (I), each R^{S1} is independently selected from —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).

Preferably, each R^{S1} is independently selected from —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), and —N(C₁₋₆ alkyl)-CO(C₁₋₆ alkyl).

More preferably, each R^{S1} is independently selected from —OH, —SH, —NH₂, —NH—OH, -HaI, —CN, —NO₂, —CHO, —COOH, and —CO—NH₂.

Even more preferably, each R^{S1} is independently selected from —OH, —SH, —NH₂, -HaI, and —CN.

In formula (I), each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), —SO—(C₁₋₆ alkyl), —(C₀₋₄ alkylene)-carbocyclyl, and —(C₀₋₄ alkylene)-heterocyclyl, wherein the carbocyclyl group in said-(C₀₋₄ alkylene)-carbocyclyl and the heterocyclyl group in said-(C₀₋₄ alkylene)-heterocyclyl are each optionally substituted with one or more groups independently selected from C₁₋₆ alkyl, C₂₋₆ alkenyl, C₂₋₆ alkynyl, halogen, C₁₋₆ haloalkyl, —O—(C₁₋₆ haloalkyl), —CN, —OH, —O(C₁₋₆ alkyl), —SH, —S(C₁₋₆ alkyl), —NH₂, —NH(C₁₋₆ alkyl), —N(C₁₋₆ alkyl) (C₁₋₆ alkyl), —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).

Preferably, each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).

More preferably, each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), and —N(C₁₋₆ alkyl)-CO(C₁₋₆ alkyl).

Even more preferably, each R^{S2} is independently selected from OH, —SH, —NH₂, —NH—OH, -HaI, —CN, —NO₂, —CHO, —COOH, and —CO—NH₂.

Even more preferably, each R^{S2} is independently selected from —OH, —SH, —NH₂, -HaI, and —CN.

In a first specific embodiment, R¹ is phenyl optionally substituted with —O(C₁₋₆ alkyl). In this first specific embodiment, R², each R³, R^{S1} and R^{S2} are as defined hereinabove for formula (I). Preferably, R¹ is 4-ethoxyphenyl.

In a second specific embodiment, R² is C₂₋₄ alkylene, optionally substituted with one or more R^{S1}. In this second specific embodiment, R¹, each R³, R^{S1} and R^{S2} are as defined hereinabove for formula (I). Preferably, R² is C₂₋₄ alkylene, preferably selected from —CH₂CH₂—, —CH₂CH₂CH₂—, —CH₂CH₂CH₂CH₂—, and —CH₂CH(CH₃)CH₂—, more preferably selected from —CH₂CH₂CH₂—, and —CH₂CH(CH₃)CH₂—, even more preferably R² is-CH₂CH₂CH₂—.

In a third specific embodiment, each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said —(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2}. In this third specific embodiment, R¹, R², R^{S1} and R^{S2} are as defined hereinabove for formula (I). Preferably, each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said-(C₀₋₃ alkylene)-aryl are each optionally substituted with one or more R^{S2}. More preferably, each R³ is independently selected from methyl, ethyl, propyl, isopropyl, butyl, isobutyl, cyclohexyl and benzyl. Further preferred combination of both R³ groups as encompassed by third embodiment of the present invention are recited in the following Table 1.

TABLE 1
Specific combinations of the two R³ groups
as encompassed by the invention.

| First R³ group | Second R³ group |
|---|---|
| methyl | methyl |
| ethyl | ethyl |
| n-butyl | methyl |
| n-butyl | ethyl |
| propyl | propyl |
| isobutyl | isobutyl |
| n-butyl | benzyl |
| methyl | cyclohexyl |

In a fourth specific embodiment of the present invention, both R³ are joined together to form together with the N atom that otherwise carries both R³ a heterocyclic ring, optionally substituted with one or more R^{S2}. In this fourth specific embodiment, R¹, R², R^{S1} and R^{S2} are as defined hereinabove for formula (I). Preferably, the heterocyclic ring optionally substituted with one or more R^{S2} is a heterocyclic ring optionally substituted with one or more C₁₋₆ alkyl groups. Preferably, the heterocyclic ring is selected from piperidine, pyrrolidine and azepane, more preferably the heterocyclic ring is selected from piperidine and pyrrolidine, even more preferably the heterocyclic ring is piperidine. Accordingly and preferably, in this fourth specific embodiment of the invention the —NR³R³ is selected from:

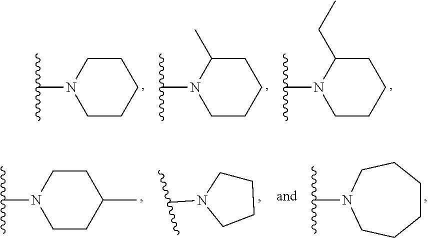

preferably selected from

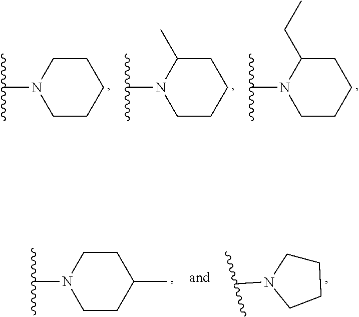

more preferably selected from

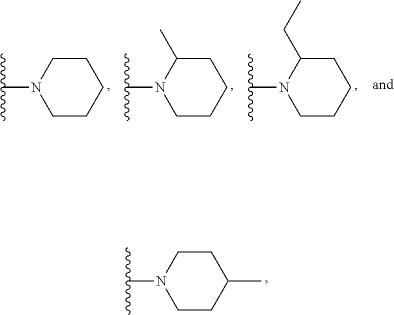

even more preferably the —NR³R³ is

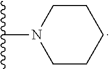 [SMILES: *N1CCCCC1]

In a fifth specific embodiment of the present invention, —R²—NR³R³ are taken together to form:

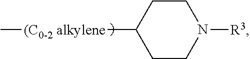

wherein R³ is selected from C₁₋₅ alkyl, C₂₋₅ alkenyl, C₂₋₅ alkynyl, (C₀₋₃ alkylene)-cycloalkyl, (C₀₋₃ alkylene)-heterocycloalkyl, (C₀₋₃ alkylene)-aryl and (C₀₋₃ alkylene)-heteroaryl, wherein said alkyl, said alkenyl, said alkynyl and the alkylene moiety in said —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said-(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2}. In this fifth specific embodiment, R¹, R^{S1} and R^{S2} are as defined hereinabove for formula (I). Preferably, said C₀₋₂ alkylene in this fifth embodiment is absent. Accordingly, preferably in this fifth specific embodiment of the present invention R²—NR³R³ are taken together to form:

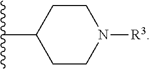

Preferably, R³ is selected from methyl, ethyl, propyl, isopropyl, butyl, isobutyl, cyclohexyl and benzyl.

It is to be understood that all the features of the compound of formula (I), as described hereinabove, can be combined with each other, unless explicitly indicated to the contrary.

In one particularly preferred embodiment, the present invention relates to a compound of formula (I):

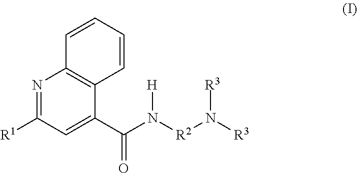

or its pharmaceutically acceptable salt, wherein:
R¹ is 4-ethoxyphenyl;
R² is C₂₋₄ alkylene; and
both R³ are joined together to form together with the N atom that otherwise carries both
R³ a heterocycloalkyl ring optionally substituted with one or more R^{S2}.

In connection with this particularly preferred embodiment, it is preferred that R² is C₃₋₄ alkylene, in particular-CH₂CH₂CH₂—.

In another particularly preferred embodiment, the present invention relates to a compound of formula (I):

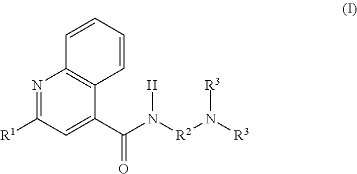

or its pharmaceutically acceptable salt, wherein:
R¹ is 4-ethoxyphenyl;
R² is C₂₋₄ alkylene; and
both R³ are joined together to form together with the N atom that otherwise carries both
R³ a heterocyclic ring optionally substituted with one or more C₁₋₆ alkyl groups.

In this particularly preferred embodiment of the invention, R² is preferably selected from —CH₂CH₂—, —CH₂CH₂CH₂—, —CH₂CH₂CH₂CH₂—, and —CH₂CH(CH₃)CH₂—, more preferably selected from —CH₂CH₂CH₂—, and —CH₂CH(CH₃)CH₂—, even more preferably R² is-CH₂CH₂CH₂—. Further, R² is preferably C₃₋₄ alkylene, in particular-CH₂CH₂CH₂—.

In this particularly preferred embodiment, the heterocyclic ring is preferably selected from piperidine, pyrrolidine and azepane, more preferably the heterocyclic ring is selected from piperidine and pyrrolidine, even more preferably the heterocyclic ring is piperidine. Accordingly and preferably, in this particularly preferred embodiment of the invention the —NR³R³ is selected from:

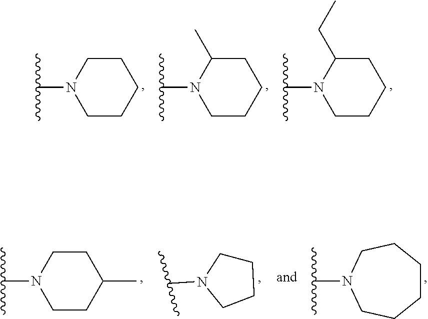

preferably selected from

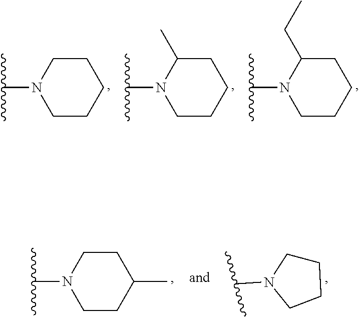

more preferably selected from

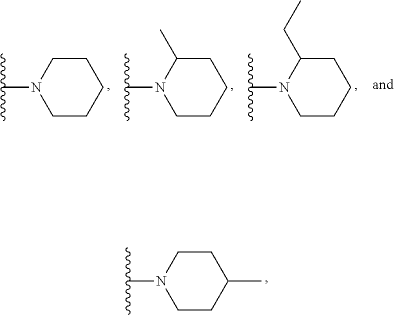

even more preferably the —NR³R³ is

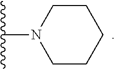 [SMILES: *N1CCCCC1]

In another particularly preferred embodiment, the present invention relates to a compound of formula (I):

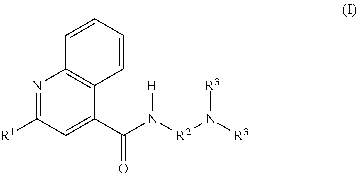

or its pharmaceutically acceptable salt, wherein:
R¹ is 4-ethoxyphenyl;
R² is C₂₋₄ alkylene; and
each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said-(C₀₋₃ alkylene)-aryl are each optionally substituted with one or more R^{S2}, wherein R^{S2} is as defined above.

In connection with this particularly preferred embodiment, it is preferred that the proviso applies that not both R³ are unsubstituted methyl and that the proviso applies that not both R³ are unsubstituted ethyl.

Further, in connection with this particularly preferred embodiment, it is preferred that R² is C₃₋₄ alkylene, in particular-CH₂CH₂CH₂—.

Particularly preferred compounds of formula (I) are selected from the following compounds or pharmaceutically acceptable salts thereof:

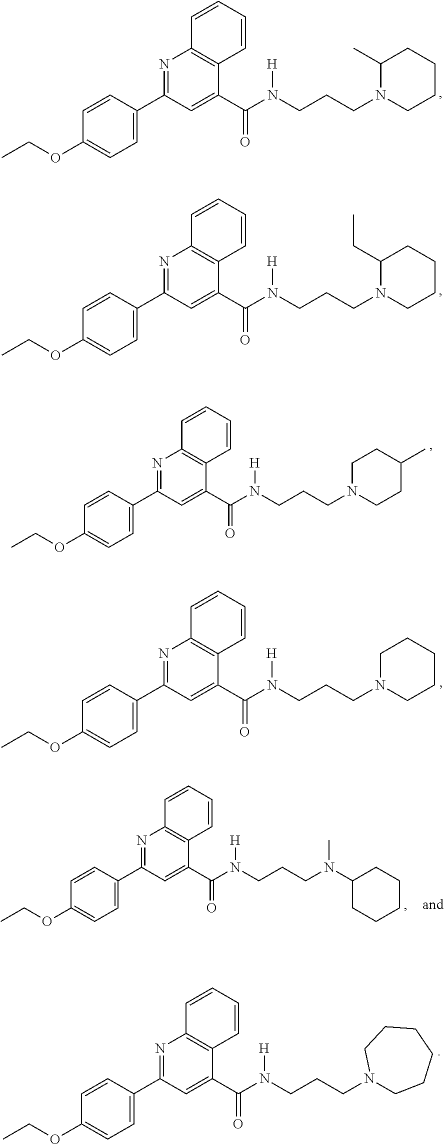

Further preferred compounds of formula (I) are selected from the following compounds or pharmaceutically acceptable salts thereof:

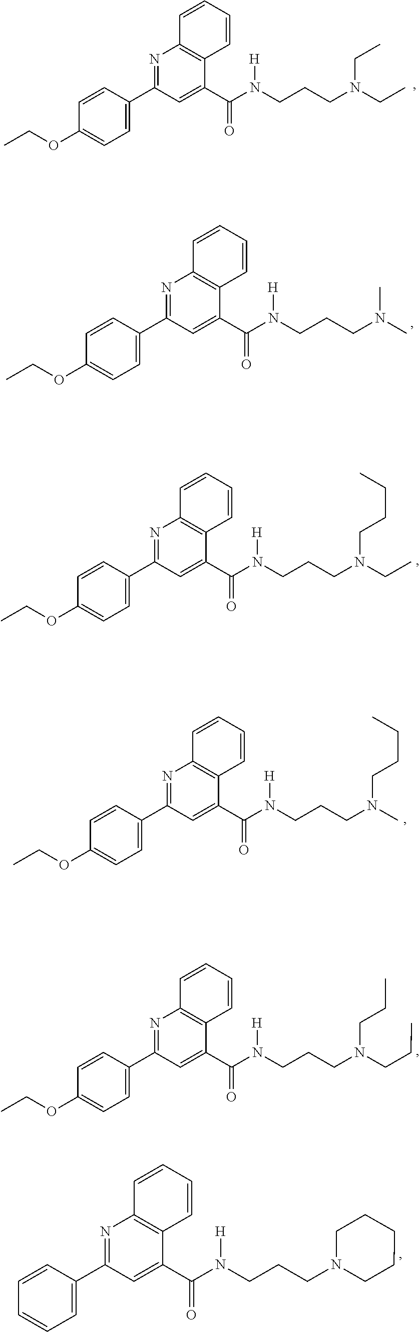

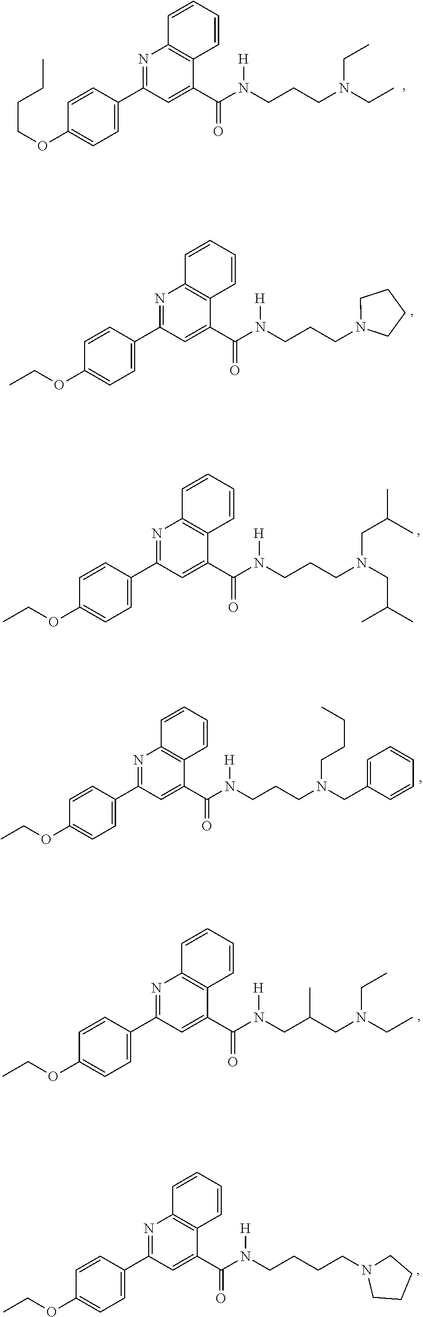

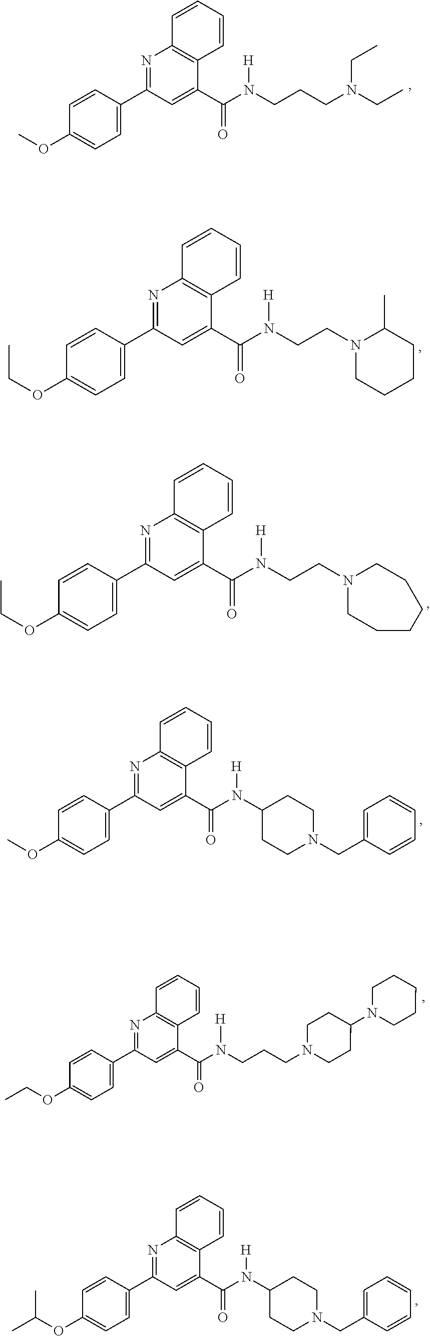

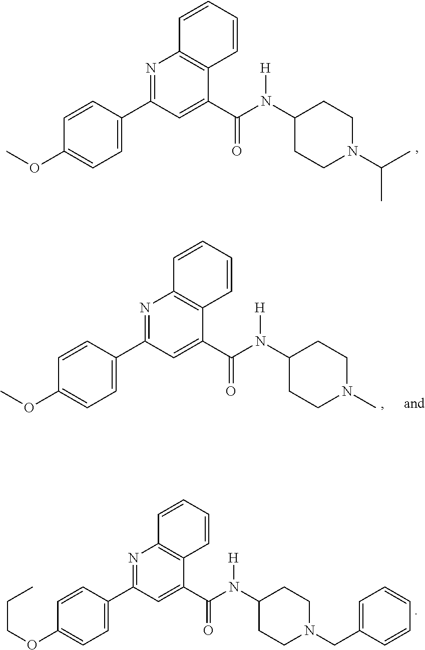

In one specific embodiment of the invention, the following compounds:

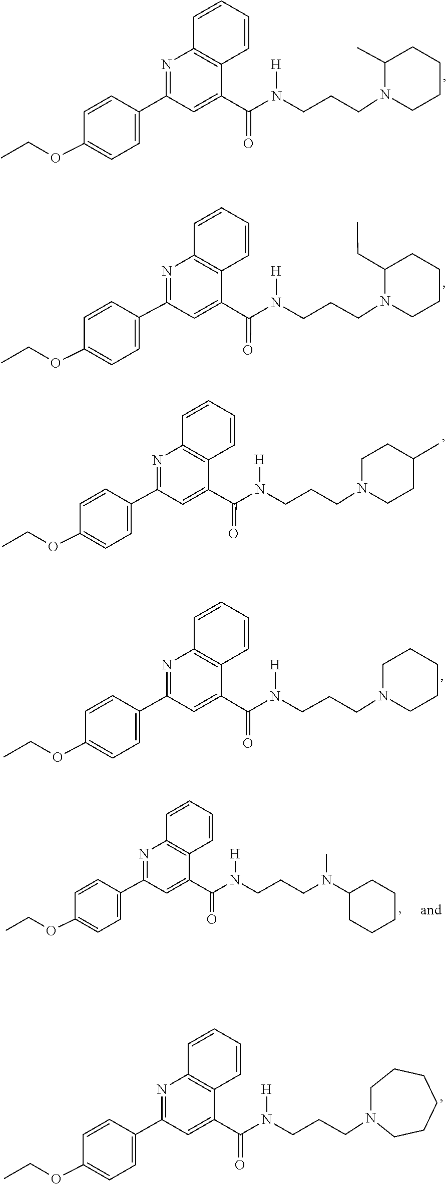

are excluded from formula (I).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N, N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved (i.e., separated) by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William JS et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds of the present invention (i.e., the compounds of formula (I)) are obtainable by the means of chemical synthesis, in a method analogous to the synthesis of dTASL1 compound, shown in Example 4.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The therapeutic use of the compounds of the present invention will be described in the following.

In one embodiment, the present invention relates to the compound of formula (I) or a pharmaceutically acceptable salt thereof for use as a medicament.

Without being bound by the theory, the present inventors have demonstrated that, as TASL is regulated by proteostatic interaction with SLC15A4, the treatment with the compound of formula (I) leads to efficient catalytic degradation of TASL protein.

It is known to the skilled person and apparent from the literature that SLC15A4 and/or TASL play a clear role in autoimmune diseases (including systemic lupus erythematosus) and inflammatory conditions (including inflammatory bowel disease, psoriasiform dermatitis and endosomal TLR-dependent inflammation.

Accordingly, there is a body of evidence based on genetic studies of systemic lupus erythematosus (SLE) in human cells indicating the role SLC15A4 in this disorder. Bentham et al. (PMID: 26502338) identified through GWAS the genetic association of SLC15A4 and TASL (referenced therein as CXorf21) with SLE in a cohort of European ancestry. Han and coworkers (PMID: 19838193) identified through GWAS the genetic association of SLC15A4 with SLE in a cohort of a Chinese Han population. He CF and coworkers (PMID: 20516000) identified through GWAS the genetic association of SLC15A4 with SLE-related discoid rash in a Chinese Han population. Langefeld and coworkers (PMID: 28714469) identified through GWAS the genetic association of SLC15A4 with SLE in a cohort of European ancestry.

Further, there is a body of evidence based on genetic studies of systemic lupus erythematosus (SLE) in human cells indicating the role TASL in this disorder. Bentham et al. (PMID: 26502338) identified through GWAS the genetic association of SLC15A4 and TASL (referenced therein as CXorf21) with SLE in a cohort of European ancestry. Odhams et al. (PMID: 31092820) proposed that genetic variants of TASL (CXorf21) associated with SLE lead to increased TASL expression in an interferon- and sex-specific manner and hence suggest a potential explanation.

There is further a body of evidence in mouse models indicating the importance of TASL/SLC15A4 in systemic lupus erythematosus. Baccala and coworkers (PMID: 23382217) identified a protective role for SLC15A4 deficiency in the development of SLE in a mouse model using the C57BL/6-Fas(lpr) strain. Kobayashi and coworkers (PMID: 25238095) show that SLC15A4-deficiency is protective in two models of SLE (pristane-induced and C57BL/6lpr/lpr mice) and that SLC15A4 is required in B cells for endosomal TLR function. Pollard and coworkers (PMID: 29055005) find that SLC15A4-deficiency is protective in a mercury-induced model of SLE. Katewa and coworkers (PMID: 33444326) describe a protective role for SLC15A4 in pristane-induced and NZB/W F1 murine genetic models of SLE.

Accordingly, the compounds of the present invention are considered useful in the treatment or prevention of autoimmune disorders, in particular systemic lupus erythematosus.

It can be further assumed, based on the role of SLC15A4 and/or TASL in IRF5 activation, that the compounds of the present invention will be useful in the treatment of prevention of autoimmune disorders, preferably selected from systemic lupus erythematosus, rheumatoid arthritis, scleroderma, Sjögren's syndrome, polymyositis/dermatomyositis, primary biliary cirrhosis, multiple sclerosis, oral ulcers, periodontitis, Behçet's disease, myasthenia gravis, and ankylosing spondylitis.

There is further a body of evidence based on genetic studies indicating the role of SLC15A4 and/or TASL in inflammatory conditions. Heinz and coworkers (PMID: 32433612) have shown essential role of SLC15A4-TASL and their relationship in human cell lines and primary cells for endosomal TLR function.

There is further a body of evidence based on the studies in mouse models indicating the role of SLC15A4 and/or TASL in inflammatory conditions. Blasius and coworkers (PMID: 21045126) identified SLC15A4 as essential component in endosomal TLR function in plasmacytoid dendritic cells. Sasawatari and coworkers (PMID: 21277849) have found SLC15A4-deficiency to impair CpG-induced production of inflammatory cytokines from dendritic cells. Furthermore, SLC15A4-deficiency was found to be protective in a mouse model of inflammatory bowel disease. SLC15A4-deficient mice also showed defective cytokine production upon activation of the NOD-like receptor NOD1. Blasius and coworkers (PMID: 23028315) suggested an important role for SLC15A4 in pDCs in controlling viral persistence, as evidenced in a model of LCMV infection. Nakamura and coworkers (PMID: 24695226) proposed a role for SLC15A4 in the activation of NOD2 at the lysosome. Of note, NOD2 mutations are linked to inflammatory bowel disease. Dosenovic and coworkers (PMID: 25310967) find defective endosomal TLR function in pDCs, splenic cDCs and B cells. Furthermore, production of antibodies elicited by a CpGA-adjuvanted vaccine is impaired in SLC15A4 mice. Griffith and coworkers (PMID: 30262916) describe a requirement for SLC15A4 in imiquimod-induced systemic inflammation and psoriasiform inflammation in mice. Lopez-Haber and coworkers (PMID: 36031853) describe a contribution of SLC15A4 in inflammasome activation via mTORC1 signaling pathways.

It can be further assumed, based on the role of SLC15A4 and/or TASL in IRF5 activation, that the compounds of the present invention will be useful in the treatment of prevention of inflammatory condition, preferably selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis, preferably wherein the inflammatory condition is selected from inflammatory bowel disease, psoriasiform dermatitis, and endosomal TLR-dependent inflammation.

Accordingly, the compounds of the present invention are useful in treatment or prevention of an inflammatory condition.

Thus, in one embodiment, the present invention relates to the compound of formula (I) or its pharmaceutically acceptable salt, or the pharmaceutical composition of the present invention, for use in treatment or prevention of an autoimmune disorder or an inflammatory condition. In one embodiment, the present invention relates to the compound of formula (I) or its pharmaceutically acceptable salt, or the pharmaceutical composition of the present invention, for use in treatment or prevention of an autoimmune disorder. In one embodiment, the present invention relates to the compound of formula (I) or its pharmaceutically acceptable salt, or the pharmaceutical composition of the present invention, for use in treatment or prevention of an inflammatory condition.

The autoimmune disorder is preferably selected from systemic lupus erythematosus, rheumatoid arthritis, scleroderma, Sjögren's syndrome, polymyositis/dermatomyositis, primary biliary cirrhosis, multiple sclerosis, oral ulcers, periodontitis, Behçet's disease, myasthenia gravis, and ankylosing spondylitis. More preferably the autoimmune disorder is systemic lupus erythematosus.

The inflammatory condition is preferably selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis. More preferably, the inflammatory condition is selected from inflammatory bowel disease, psoriasiform dermatitis, and endosomal TLR-dependent inflammation.

In one embodiment, the present invention relates to use of the compound of formula (I) or its pharmaceutically acceptable salt, or the pharmaceutical composition of the present invention, in the manufacture of a medicament for use in the treatment or prevention of an autoimmune disorder or an inflammatory condition. In one embodiment, the present invention relates to use of the compound of formula (I) or its pharmaceutically acceptable salt, or the pharmaceutical composition of the present invention, in the manufacture of a medicament for use in the treatment or prevention of an autoimmune disorder. In one embodiment, the present invention relates to use of the compound of formula (I) or its pharmaceutically acceptable salt, or the pharmaceutical composition of the present invention, in the manufacture of a medicament for use in the treatment or prevention of an inflammatory condition.

In one embodiment, the present invention relates to the method of treating an autoimmune disorder, the method comprising administering the compound of formula (I) or its pharmaceutically acceptable salt, or the pharmaceutical composition of the present invention, to a subject in need thereof. It is to be understood that said compound or its salt or said pharmaceutical composition is to be administered in a therapeutically effective amount, preferably as described herein.

In one embodiment, the present invention relates to the method of treating an inflammatory condition, the method comprising administering the compound of formula (I) or its pharmaceutically acceptable salt, or the pharmaceutical composition of the present invention, to a subject in need thereof. It is to be understood that said compound or its salt or said pharmaceutical composition is to be administered in a therapeutically effective amount, preferably as described herein.

It is to be noted that the compound of formula (I) or its salt has been shown by the present inventors to inhibit SLC15 peptide transporter (i.e., SLC15A4). Accordingly and preferably, it is expected that the therapeutic effect of the compound of formula (I) or its salt is based on the inhibition of SLC15 peptide transporter (i.e., SLC15A4). Accordingly, in one embodiment the present invention relates to the compound of formula (I) or its salt, or the pharmaceutical composition of the present invention for use in the treatment or prevention of an autoimmune disorder, wherein said compound or said pharmaceutical composition inhibits SLC15 peptide transporter. Given the ability of the compound of the present invention, or its salt, to inhibit the SLC15 peptide transporter, said compound is particularly useful in the treatment of the autoimmune disorder, wherein the autoimmune disorder is a disorder associated with SLC15 peptide transporter. Accordingly, the present invention in one embodiment relates to the compound of formula (I) or its salt, or the pharmaceutical composition of the present invention wherein said compound or said pharmaceutical composition for use in the treatment or prevention of an autoimmune disorder, wherein the autoimmune disorder is a disorder associated with SLC15 peptide transporter.

The compounds provided in the present invention are also useful in treatment or prevention of other lupus diseases such as cutaneous or neonatal lupus erythematosus.

Further embodiments of the present invention are disclosed in the following numbered items.
1. A compound of formula (I):

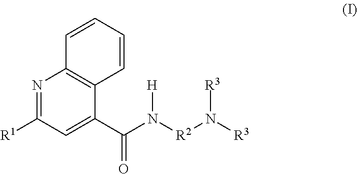

or a pharmaceutically acceptable salt thereof, wherein:
R¹ is aryl or heteroaryl, optionally substituted with one or more R^{S2},
R² is C₁₋₅ alkylene, optionally substituted with one or more R^{S1},
each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said —(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2}, or
both R³ are joined together to form together with the N atom that otherwise carries both R³ a heterocyclic ring, optionally substituted with one or more R^{S2}, or —R²—NR³R³ are taken together to form:

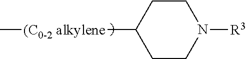

wherein R³ is selected from C₁₋₅ alkyl, C₂₋₅ alkenyl, C₂₋₅ alkynyl, (C₀₋₃ alkylene)-cycloalkyl, (C₀₋₃ alkylene)-heterocycloalkyl, (C₀₋₃ alkylene)-aryl and (C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said —(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2};
wherein each R^{S1} is independently selected from —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl); and wherein each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), —SO—(C₁₋₆ alkyl), —(C₀₋₄ alkylene)-carbocyclyl, and —(C₀₋₄ alkylene)-heterocyclyl, wherein the carbocyclyl group in said-(C₀₋₄ alkylene)-carbocyclyl and the heterocyclyl group in said-(C₀₋₄ alkylene)-heterocyclyl are each optionally substituted with one or more groups independently selected from C₁₋₆ alkyl, C₂₋₆ alkenyl, C₂₋₆ alkynyl, halogen, C₁₋₆ haloalkyl, —O—(C₁₋₆ haloalkyl), —CN, —OH, —O(C₁₋₆ alkyl), —SH, —S(C₁₋₆ alkyl), —NH₂, —NH(C₁₋₆ alkyl), —N(C₁₋₆ alkyl) (C₁₋₆ alkyl), —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).
2. The compound of item 1, wherein R¹ is phenyl optionally substituted with one or more R^{S2}, preferably wherein R¹ is phenyl substituted with —O(C₁₋₆ alkyl).
3. The compound of item 1 or 2, wherein R¹ is 4-ethoxyphenyl.
4. The compound of any one of items 1 to 3, wherein each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said —(C₀₋₃ alkylene)-aryl are each optionally substituted with one or more R^{S2}.
5. The compound of item 4, wherein each R³ is independently selected from methyl, ethyl, propyl, isopropyl, butyl, isobutyl, cyclohexyl and benzyl.
6. The compound of any one of items 1 to 3, wherein both R³ are joined together to form together with the N atom that otherwise carries both R³ to form a heterocyclic ring optionally substituted with one or more R^{S2}.
7. The compound of item 6, wherein the heterocyclic ring optionally substituted with one or more R^{S2} is piperidine ring or pyrrolidine ring, optionally substituted with one or more R^{S2}, preferably a 2-methylpiperidine ring.
8. The compound of any one of item 1 to 3, —R²—NR³R³ are taken together to form:

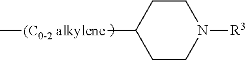

preferably wherein R³ is selected from methyl, ethyl, propyl, isopropyl, butyl, isobutyl, cyclohexyl and benzyl.
9. The compound of any one of item 1 to 8, wherein the following compounds:

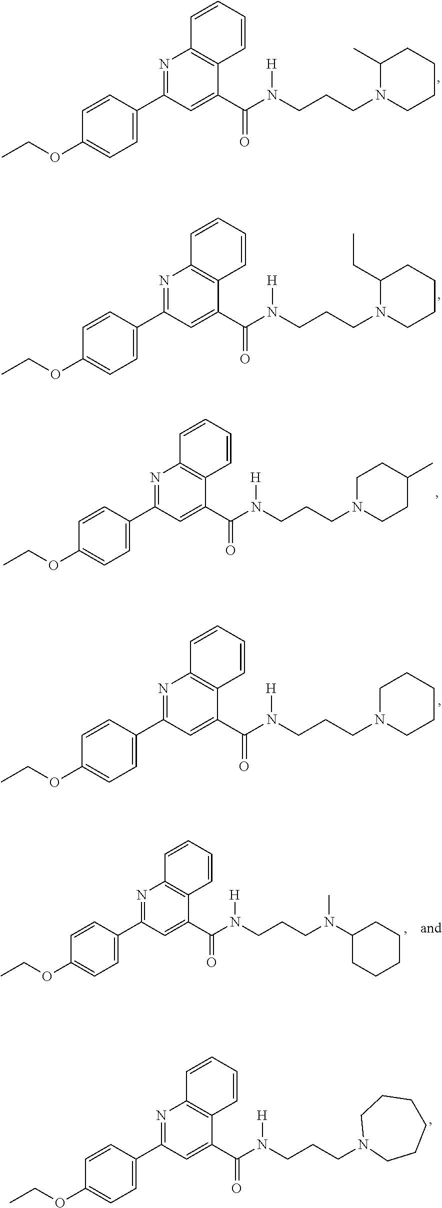

are excluded from formula (I).
10. The compound of item 1, wherein the compound is a compound selected from the following compounds or their pharmaceutically acceptable salts:

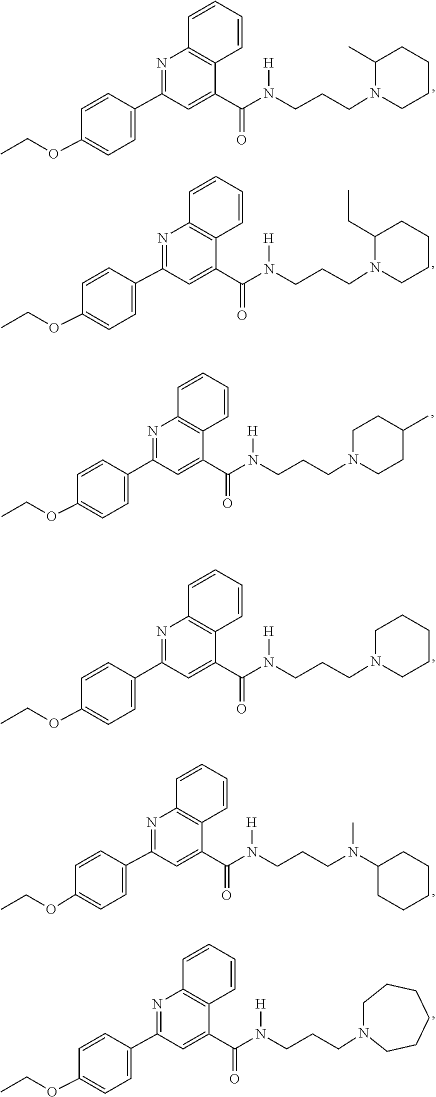

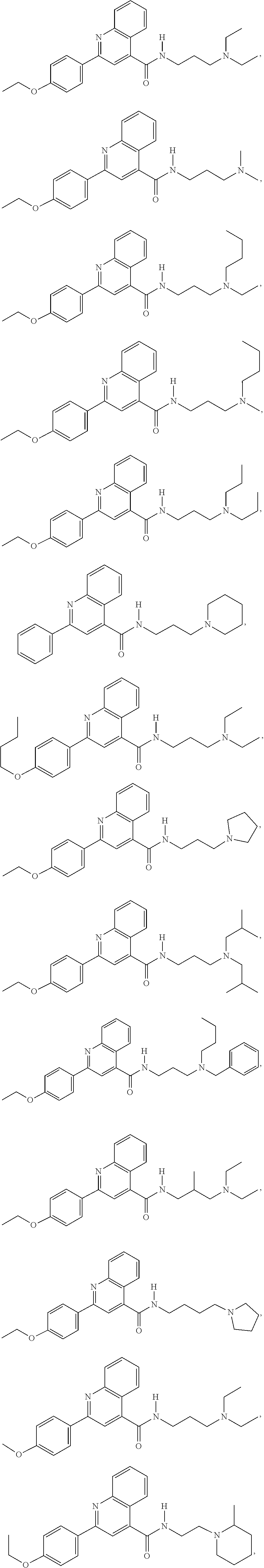

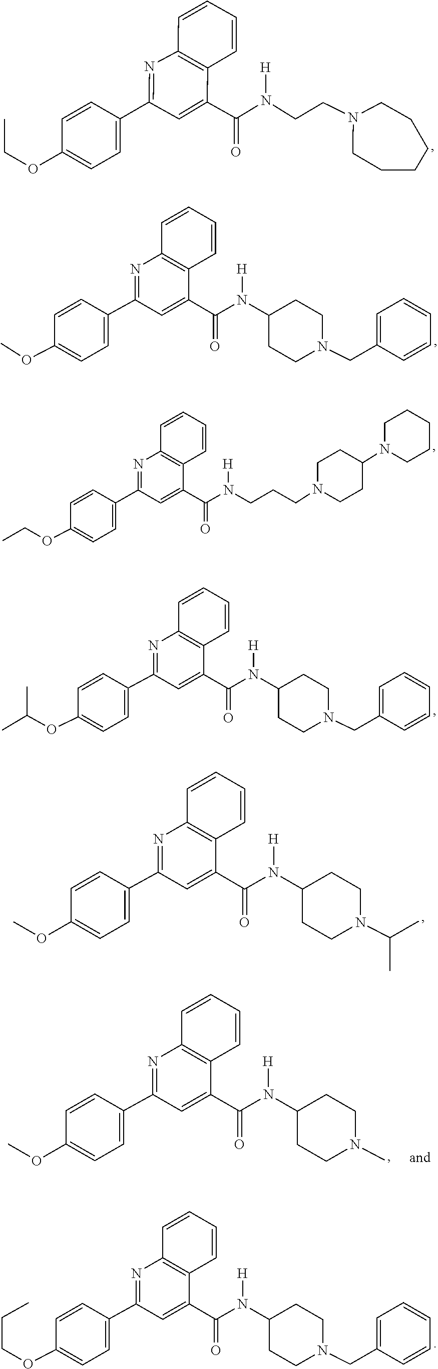

11. A pharmaceutical composition comprising the compound of any one of items 1 to 10 and at least one pharmaceutically acceptable carrier.
12. The compound of any one of items 1 to 10 or the pharmaceutical composition of item 11 for use as a medicament.
13. The compound of any one of items 1 to 10 or the pharmaceutical composition of item 11 for use in treatment or prevention of an autoimmune disorder or inflammatory condition.
14. The compound for use or the pharmaceutical composition for use of item 13, wherein the autoimmune disorder is selected from systemic lupus erythematosus, rheumatoid arthritis, scleroderma, Sjögren's syndrome, polymyositis/dermatomyositis, primary biliary cirrhosis, multiple sclerosis, oral ulcers, periodontitis, Behçet's disease, myasthenia gravis, and ankylosing spondylitis, preferably wherein the autoimmune disorder is systemic lupus erythematosus, or
wherein the inflammatory condition is selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis, preferably wherein the inflammatory condition is selected from inflammatory bowel disease, psoriasiform dermatitis, and endosomal TLR-dependent inflammation.
15. The compound for use or the pharmaceutical composition for use of item 13 or 14, wherein the autoimmune disorder is a disorder associated with SLC15 peptide transporter, and/or wherein said compound or said pharmaceutical composition inhibits SLC15 peptide transporter.

The invention will be illustrated in the following examples, which however are not to be construed as limiting.

### EXAMPLES

### Materials and Methods

### Antibodies and Reagents

Custom rabbit anti-SLC15A4 antibodies were generated by Genscript (raised against the N-terminus of SLC15A4). Custom rabbit anti-TASL antibodies (TASL HM) were produced by Eurogentech (recognizing the C-terminus of TASL); anti-CXorf21 (TASL) (HPA00118) antibody was from Sigma; anti-IRF5 (EPR17067) rabbit mAB (ab181553) from abcam; anti-LAMP1 (H4A3) (sc-20011) and anti-GAPDH (G-9) mouse mABs (sc-365062) from Santa Cruz; anti-SAPK/JNK antibody (no. 9252), anti-IkBα (L35A5) mouse mAB (no. 4814), anti-phospho-SAPK/JNK Thr183/Tyr185 (81E11) (no. 4668), anti-STAT1 (D1K9Y) (no. 14994), anti-phospho-STAT1 (Y701) (no. 7649), anti-phospho-IkBα Ser32 (14D4) (no. 2859), anti-NF-κB p65 (D14E12) (no. 8242), anti-phospho-NF-κB p65 Ser536 (93H1) (no. 3033), anti-HA (C29F4) (no. 3724), anti-GFP (no. 2956), anti-V5 (D3H8Q) (no. 13202) and directly conjugated anti-IRF5-Alexa647 (no. 74818) and anti-NF-κB p65-Alexa488 (no. 49445) rabbit mAbs were from Cell Signaling. PE-anti-CD19 was from BioLegend (no. 302254). Anti-rabbit IgG (H+L), F(ab′)2 Fragment (Alexa Fluor. 647 Conjugate) secondary antibodies (no. 4414) were from Cell Signaling. R⁸⁴⁸ and Pam3CKS4 were from Invivogen.

### Chemical Compounds

dTASL1 and dTASL1 analogs were purchased from ChemDiv, Inc. Other early lead molecules were acquired from Vitas M Chemical Limited (C1), ChemBridge (C8), and Chesmpace LLC (C10 and C4). Chemical synthesis of dTASL1 is described in Example 4.

### Cell Culture and Human Primary Cells

HEK293T cells and THP1 cells were purchased from ATCC, THP1 DUAL/ISRE reporter cell lines from Invivogen. CAL-1 cells were provided by T. Maeda (Nagasaki University). Cell lines—except for CAL-1—were authenticated by short tandem repeat profiling and regularly tested for mycoplasma contamination. Human primary PBMCs were isolated from the blood of anonymous healthy donors as well as SLE patients included in our biobank approved by the ethics committee of the Medical University of Vienna (EK2071/2020 and EK1075/2021). CD14⁺ monocytes were purified by magnetic-activated cell sorting (Miltenyi Biotec, #130-050-201) according to the manufacturers protocol. Purity of CD14⁺ cells ranged between 80.8% and 94.2% as determined by flow cytometry analysis. Hek293T cells were cultured in DMEM, THP1, CAL-1, primary human PBMCs and CD14⁺ monocytes in RPMI, supplemented with 10% (v/v) FBS and antibiotics (100 U/ml penicillin, 100 g/ml streptomycin), all from Gibco. Cells were incubated at 37 °C in 5% CO2.

Plasmids and siRNAs

Codon-optimized cDNAs for human wildtype and mutant SLC15A4 and TASL were obtained from Genscript and sub-cloned into pDONR201 (Invitrogen) plasmids as reported previously. The vector required for creation of the TGC cell line was assembled from a gateway (gw) compatible pRRL-EF1-gw-EmGFP-IRES-HygroR vector and a vector containing the P2A-mCherry sequence. The human codon-optimized TASL sequence was then subcloned into the resultant gateway compatible vector. For lentiviral transduction, cDNAs were shuttled to pRRL-based lentiviral expression plasmids and a previously described selectable resistance cassette¹. Lentiviral packaging plasmids psPAX2 (plasmid no. 12260) and pMD2.G (plasmid no. 12259) were obtained from Addgene. Full length TASL and shorter constructs (residues 1-8, 1-15, 1-94 and 1-193) were cloned into a pDONR221 vector without a stop codon and further subcloned into a pLIX403 plasmid with a C-terminal V5-tag (Addgene no. 41395). Fusions of TASL N-terminal sequences (aa 1-12, 1-13, 1-14 and 1-15) with GFP were generated by introducing corresponding annealed oligos into the BsmBI sites of the Artichoke reporter vector (Addgene plasmid no. 73320) via Golden Gate assembly. The used oligo sequences were as follows: TASL 1-12F: 5′-ccatGCTGAGCGAGGGCTATCTGAGCGGACTGGAGTAT-3′ (SEQ ID NO: 1), TASL 1-12R: 5′-cagcATACTCCAGTCCGCTCAGATAGCCCTCGCTCAGC-3′ (SEQ ID NO: 2); TASL 1-13F: 5′-ccatGCTGAGCGAGGGCTATCTGAGCGGACTGGAGTATTGG-3′ (SEQ ID NO: 3), TASL 1-13R: 5′-cagcCCAATACTCCAGTCCGCTCAGATAGCCCTCGCTCAGC-3′ (SEQ ID NO: 4); TASL 1-14F: 5′-ccatGCTGAGCGAGGGCTATCTGAGCGGACTGGAGTATTGGAAC-3 (SEQ ID NO: 5), TASL 1-14R: 5′-cagcGTTCCAATACTCCAGTCCGCTCAGATAGCCCTCGCTCAGC-3′ (SEQ ID NO: 6); TASL 1-15F: 5′-ccatGCTGAGCGAGGGCTATCTGAGCGGACTGGAGTATTGGAACGAC-3′ (SEQ ID NO: 7), TASL 1-15R: 5′-cagcGTCGTTCCAATACTCCAGTCCGCTCAGATAGCCCTCGCTCAGC-3′ (SEQ ID NO: 8). LAMTOR1 (1:81)-TASL fusion was cloned by Gibson assembly into a pRRL-based lentiviral expression plasmid. siRNAs targeting codon optimized human SLC15A4 were obtained from Dharmacon. Non-targeting pool, control (cat.: D-001810 Oct. 5), was reported previously (21) and codon optimized human SLC15A4 targeting siRNA no. 1: 5′-GGGCAGCCUUCUACGGAAUUU-3′ (SEQ ID NO: 9) and no. 2: 5′-CAACCAGACCAUCGGCAAUUU-3′ (SEQ ID NO: 10). siRNA transfection was performed with a reverse transfection protocol using RNAiMX Lipofectamine (ThermoFisher). sgRNA used have been previously described (21): cloned oligonucleotides were as follows (5′ to 3′ orientation):

-SLC15A4 sgRNA no. 1,
F:
(SEQ ID NO: 11)
CACCGGGAGCGATCCTGTCGTTAGG,
R:
(SEQ ID NO: 12)
AAACCCTAACGACAGGATCGCTCCC
-SLC15A4 sgRNA no. 2,
F:
(SEQ ID NO: 13)
CACCGTATTACAACCACTCCTCACA,
R:
(SEQ ID NO: 14)
AAACTGTGAGGAGTGGTTGTAATAC
-TASL sgRNA no. 1,
F:
(SEQ ID NO: 15)
CACCGGTAGAAATGGAATCCTCCAT,
R:
(SEQ ID NO: 16)
AAACATGGAGGATTCCATTTCTACC
-TASL sgRNA no. 2,
F:
(SEQ ID NO: 17)
CACCGCTGAATTAATGGCCATCACC,
R:
(SEQ ID NO: 18)
AAACGGTGATGGCCATTAATTCAGC

### Lentiviral Transduction

For lentiviral gene transduction, Hek293T cells were transfected with the respective lentiviral vectors and packaging plasmids psPAX2 and pMD2.G using PEI (Sigma). Twenty-four hours later, medium was exchanged to DMEM, supplemented with 10% (v/v) FBS and antibiotics (100 U/ml penicillin, 100 μg/ml streptomycin). Seventy-two hours after transfection, cell supernatants were collected, filtered through 0.45-μm polyethersulfone filters (GE Healthcare) and supplemented with 8 μg/ml protamine sulfate (Sigma). Media above newly plated Hek293T cells was exchanged for the virus containing protamine sulfate supplemented supernatants. Twenty-four hours after infection, medium was changed; forty-eight hours after infection, cells were selected with the respective antibiotics.

### Compound Treatment and R848 Stimulation

CAL-1, THP1 or human primary cells were incubated with dTASL1 and other identified compounds (or the corresponding volume of DMSO vehicle) for 24 h or 48 h at the indicated concentration and subsequently stimulated with R848 (5 μg/ml) for the indicated time. Supernatant or cells were recovered for ELISA, SDS-PAGE or microscopy-based analysis.

### Cell Lysis and Western Blotting

Cells were lysed in RIPA lysis buffer (25 mM Tris, 150 mM NaCl, 0.5% NP-40, 0.5% deoxycholate (w/v) and 0.1% SDS (w/v), pH 7.4) supplemented with Roche EDTA-free protease inhibitor cocktail (one tablet for 50 mL) and halt phosphatase inhibitor cocktail (Thermo Fisher Scientific), 10 min on ice. After lysate clearing by centrifugation (13,000 rpm, 10 min, 4 °C), proteins were quantified with BCA (Thermo Fisher Scientific) using BSA as standard. Cell lysates were resolved by regular or Phos-Tag-containing (20 or 50 UM, WAKO Chemicals) SDS-PAGE. After electrophoresis, Phos-tag-containing SDS-PAGE were soaked in transfer buffer with 10 mM EDTA for 3x10 min, rinsed 10 min in transfer buffer without EDTA and blotted to nitrocellulose membranes (Amersham, Glattbrugg, Switzerland). Membranes were blocked with 5% non-fat dry milk in TBST and probed with the indicated antibodies. Binding was detected with anti-mouse-HRP secondary antibodies (no. 115-035-003) or anti-rabbit-HRP secondary antibodies (no.111-035-003), from Jackson Laboratories, using the ECL western blotting system (Advansta). When multiple antibodies were used, equal amounts of samples were loaded on multiple SDS-PAGE gels and western blots were sequentially probed with a maximum of two antibodies.

### Co-Immunoprecipitation and StrepTactin Pull-Down

For co-immunoprecipitation and StrepTactin pull-downs, 1×10⁷ THP1 cells were lysed in E1A (50 mM HEPES, 250 mM NaCl, 5 mM EDTA, 1% NP-40, pH 7.4) lysis buffer supplemented with Roche EDTA-free protease inhibitor cocktail (1 tablet per 50 ml) for 10 min on ice. Lysates were cleared by centrifugation for 10 min at 13.000 rpm, 4 °C and normalized using Bradford assay (Bio-Rad). After removal of whole cell lysate as input, the remaining material was subjected to immunoprecipitation with anti-HA agarose (Sigma) or pull-down with StrepTactin beads (IBA Lifesciences) over night at 4 °C. After washing of beads three times with E1A buffer, proteins were eluted with SDS sample buffer and analyzed by western blot.

### PNGase Treatment

Cells were lysed in RIPA buffer. Per sample, 35 μl of cleared lysate was either incubated with or without 0.5-1 μl (500-1,000 U) PNGase F (NEB) for 30 min at 37 °C. Samples were analysed by western blotting.

### Enzyme-Linked Immunosorbent Assay

All enzyme-linked immunosorbent assay (ELISA) experiments were performed using diluted cell supernatant according to manufacturer's instructions. ELISA kits for human IL-6 (no. 88-7066-88), human CCL2 (no. 88-7399-88) and human TNF (no. 88-7346-77) were from Invitrogen. ELISA kit for human interferon beta (no. 88-7066-88) was from PBL Assay Science (41410-1).

### THP1 DUAL/ISRE Cell Reporter Assay

THP1 DUAL cells (1×10⁵ cells per 96 well) were incubated with compounds for 24 h or 48 h, and subsequently stimulated for 20 h as indicated. Cell culture supernatant were collected, cleared of residual cells by centrifugation and analyzed for NF-κB and ISRE reporter activity according to the manufacturer's instructions. For experiments in 384-well format, compounds were pre-plated at defined concentrations and THP1 dual cells were added at 800 k/well concentration in 40 mL. After incubation for 24 h or 48 h cells were stimulated with 10 ml of a 25 μg/ml stock of R848 or 0.5 μg/ml Pam3CKS4 5× stocks as indicated for 20 h and detected by directly adding Quanti-Luc Plus (Invivogen) solution to the plates, according to the manufacturer's instructions and without adding stabilizer solution. Quanti-Blue detection was carried out according to the manifacturers instructions, with QB reagent and buffer (Invivogen) dissolved in 30 ml water, instead of the 100 ml used for standard detection.

### Flow Cytometry

Cells transfected with TASL-V5 plasmid were stained for V5-tag and detected with deep red Alexa Fluor 647 secondary antibody to allow for selection in the presence of an mCherry reporter. Cells were measured either immediately after staining or in the case of live cells immediately after harvesting and washing with PBS. Data were acquired on a BD FACSCalibur (BD Biosciences) and analyzed with FlowJo software (version 10).

### Confocal Microscopy

For staining of fixed cells, 12 k cells/well were seeded in tissue culture-treated PhenoPlate 96-well plates (PerkinElmer) and incubated over-night. Cells were transfected with TASL-V5 fragments the next day and subsequently TASL-V5 expression was induced by addition of 1 μg/ml doxycycline and 10 mM dTASL1 or an equivalent volume of DMSO respectively. Compound and doxycycline were added at the same time, to allow dTASL1 to occupy SLC15A4 binding sites in advance of TASL binding. Cells were fixed after 24 h of incubation for 10 min with 4% formaldehyde in PBS and permeabilized and blocked with 0.2% Triton X-100 (Sigma) and 5% FBS in PBS for 1 h. Cells were stained overnight at 4 °C with a mouse anti V5 primary antibody (Invitrogen) in blocking solution. Cells were washed 3 time in PBS and stained for 1.5 h with fluorescently labelled goat anti mouse Alexa-568 (Invitrogen) secondary antibody. After 2 more washes, nuclear counterstaining was performed for 10 min with DAPI (Thermo Fisher Scientific), diluted 1:1000 in PBS.

For live cell imaging cells were plated in PhenoPlate 96-well plates or Ibidi m-Slide 8 (Ibidi) wells at 16 k/well and incubated over-night. Then compounds were added, if indicated, and cells were imaged after 24 h of incubation. Cells were stained with LysoTracker Deep Red L12492 (ThermoFisher) and Hoechst 34580 (ThermoFisher). PhenoPlate images were acquired on an Opera Phenix Plus High-Content Screening System (PerkinElmer) and m-Slide images were acquired on a confocal laser scanning microscope (Zeiss LSM980, Carl Zeiss) equipped with an incubation chamber. Images were analyzed with Fiji-ImageJ.

### High-Throughput Screening

TGC cells were plated at 2 k/well on 384-well CellCarrier Ultra microplates preprinted with 10 mM compound in DMSO and incubated for 48 h. Plates were imaged with an Operetta CLS High Content Analysis system at one field per well and 20× magnification. A pipeline to analyze GFP intensity and mCherry/GFP ratio was set up with Operetta software.

### Microscopy-Based IRF5- and NF-κB-Specific Nuclear Translocation Assay

### CAL-1 Cells

5×10⁵ CAL-1 cells in 500 μl medium (wildtype, drug-treated or gene-deficient as indicated) were seeded on poly-L-lysine hydrobromide (P6282, Sigma-Aldrich)-coated coverslips in 24-well cell culture plates and immediately stimulated with 5 μg/ml R848 (Invivogen) or left unstimulated. Afterwards, medium was removed and cells were fixed with 4% formaldehyde in PBS for 15 minutes at room temperature (RT), followed by blocking and permeabilization with 5% FBS and 0.3% Triton X-100 in PBS for 1 h at RT. Cells were stained with directly conjugated anti-NF-κB p65-Alexa488 (Cell Signaling, no. 49445, 1:200) and anti-IRF5-Alexa647 (Cell Signaling, no. 74818, 1:200) in 1% BSA and 0.3% Triton X-100 in PBS over night at 4 °C. Slides were rinsed twice with PBS, and nuclear counterstaining was performed with DAPI (Thermo Fisher Scientific, D1306), diluted 1:2,000 in PBS. Stained slides were mounted using ProLong Gold (Thermo Fisher Scientific) antifade reagent.

Images were acquired on a Zeiss LSM-980 equipped with an AiryScan2 detector and processed in Zeiss ZEN version 3.6. Computational image analysis was performed with CellProfiler version 4.2.4 and data analyzed in R version 4.2.1 facilitating the following packages: ‘tidyverse’, ‘data.table’ and ‘ggpubr’. In brief nuclei and cytoplasmic regions were identified and nuclear to cytoplasmic median intensity ratios were calculated on a single cell level.

### Human Primary PBMCs

PBMCs were treated for 24 h with DMSO or dTASL1 (10 UM) and thereafter stimulated with 5 μg/ml R848 (Invivogen) for three hours or left unstimulated. Cells were harvested and stained for 30 minutes with PE-conjugated anti-CD19 antibodies (BioLegend, #302254, 1:200 in PBS/2% FBS) on ice and fixed with 4% formaldehyde in PBS for 15 minutes at RT. Permeabilization and staining for NF-κB p65 and IRF5 and counterstaining was performed as for CAL-1 and cells were seeded at a concentration of 3×10⁴ cells/well onto 384-well imaging plates. Images were acquired on a PerkinElmer Opera Phenix HCS spinning disc confocal microscope and analyzed as described above with the additional identification of B cells based on CD19 membrane staining intensity. This protocol also applies to the cells obtained from SLE patients.

Modeling of SLC15A4 in Complex with TASL and dTASL1

Alphafold predictions of the SLC15A4 TASL complex were generated with Alphafold-multimer and models were further evaluated visually in PyMol (Schroedinger). AutoDock Vina was used with a rigid receptor setting of the TASL-bound SLC15A4 structure predicted by Alphafold to dock dTASL1, after the bound TASL was removed from the complex structure (PMID: 19499576). AutoDockTools was used to prepare the docking parameters (PMID: 19399780). Exhaustiveness was set to ten and nine poses were acquired.

### Example 1: Screening Efforts and Small Molecule Discovery Campaign

To better explore the molecular basis of the binding of TASL to SLC15A4, the present inventors modelled the SLC15A4/TASL complex with AlphaFold2 (FIG. 1B) (J. Jumper et al., Highly accurate protein structure prediction with AlphaFold. Nature, (2021); R. Evans et al., Protein complex prediction with AlphaFold-Multimer. bioRxiv, 2021.2010.2004.463034 (2022)). While much of the TASL structure was poorly defined, in the highest ranked models, an alpha-helix at its N-terminus (formed by residues 1-23) was seen intruding deep into the peptide binding cavity of an inward open SLC15A4 conformation (top iptm score: 0.69). This was consistent with the previous interaction studies, which showed that mutation of the polar residues in the first eight amino acids of TASL abolished complex formation. Moreover, in the AlphaFold model, glutamate 465 (E465) in SLC15A4, required for TASL binding, was predicted to form a salt-bridge with the N-terminal amino-moiety of TASL. Of note, in related peptide transporters SLC15A1/2, the glutamate corresponding to E465 mediates interactions with the N-termini of the cargo peptides (M. Killer, J. Wald, J. Pieprzyk, T. C. Marlovits, C. Low, Structural snapshots of human PepT1 and PepT2 reveal mechanistic insights into substrate and drug transport across epithelial membranes. Sci Adv 7, eabk3259 (2021)). Mutation of E465 has been previously used to impair SLC15A4 transport and to support the notion that SLC15A4 transport activity is required for TLR7-9 responses. To further probe the accuracy of this model, a panel of SLC15A4 residues predicted to interact with TASL directly, as well as a few adjacent residues as controls, were selected for a focused alanine scanning mutagenesis analysis (FIG. 1C). After reconstitution of SLC15A4-deficient THP1 cells with these SLC15A4 variants, immunoprecipitation experiments revealed that mutation of residues involved in salt bridges (E465A, R48A), hydrogen bonds (Y52A) and hydrophobic interactions (M82A) with TASL profoundly reduced TASL binding. The disruption of weaker CH-TT bonds (F51A, F492A) had a reduced but detectable effect, while mutation of adjacent residues, such as E44 and E47 (which form part of the ExxER motif required for proton-coupled transport in other SLC15A4-related transporters) did not affect TASL recruitment (FIG. 1C). Lastly, it was assessed whether the N-terminal portion of TASL, that in the AlphaFold prediction intrudes into the SLC15A4 channel, was sufficient to mediate binding. SLC15A4 co-immunoprecipitated a peptide representing the first 13 amino acids of the protein, demonstrating that the N-terminus of TASL is indeed sufficient for the interaction (FIG. 1D). In contrast, SLC15A4 failed to recruit TASL, possibly because of a steric hindrance mediated by the C-terminal GFP, which has been hypothesized to prevent the shorter peptides of inserting sufficiently deep into the SLC15A4 channel. Collectively, the data were compatible with a new and unprecedented mode of binding between a protein ligand and a solute carrier. Although only a model, the extent and nature of the observed mode-of-interaction, together with the outcome of mutagenesis and interaction studies, strongly support its validity while suggesting an exquisite opportunity for chemical targeting.

The small-molecule discovery campaign as disclosed herein exploits two features: i) a potentially druggable binding interface essential for complex formation and ii) the instability of unbound TASL. The proteostatic relationship between SLC15A4 and TASL has been used by the present inventors as a basis to devise an image-based assay to monitor the interaction. A reporter construct encoding C-terminally GFP-tagged TASL, followed by the P2A self-cleaving peptide and mCherry (TASL-EmGFP-P2A-mCherry, TGC reporter), which allowed constitutive expression of both ORFs from a common promoter (FIG. 1E), has been prepared. Thus, the comparison of GFP to mCherry fluorescence enables ratiometric monitoring of TASL-GFP proteostatic stability. After generating HEK293T cells stably expressing this TGC reporter, the present inventors have observed that co-expression of SLC15A4 stabilized TASL-GFP protein levels, which is otherwise rapidly degraded. Confocal fluorescence microscopy analysis showed that TASL-GFP localized to lysosomes in presence of SLC15A4, while co-expression of a deltaN-SLC15A4, lacking the lysosomal targeting di-leucine motif, redirected the TASL-GFP signal to the plasma membrane (FIG. 1E). This confirmed that in the TGC reporter system as reported herein, TASL-GFP stabilization and localization was dependent on assembly with SLC15A4. For the chemical screening a TGC reporter cell clone stably expressing SLC15A4, characterized by a clear reduction of the TASL-GFP/mCherry ratio upon knockdown of SLC15A4 (FIGS. 1F, and 1G), has been selected. To validate the phenotypic screening set-up and ascertain its feasibility for scoring complex assembly, it was assessed whether SLC15A4-bound TASL-GFP could be displaced by defined N-terminal TASL portions. Transient overexpression of full-length C-terminally V5-tagged TASL (TASL-V5) in SLC15A4-TGC reporter cells, resulted in reduced TASL-GFP levels, likely due to competition for SLC15A4 binding (FIG. 1H). Similar reduction was observed also in cells expressing high levels of N-terminal TASL fragments, with the portion encoding the first eight residues of TASL showing a significant effect. These experiments indicated that interfering with the SLC15A4-TASL interaction could reduce complex formation to a level detectable in the assay reported herein.

Based on these results, the reporter cellular system was used to screen a diversity library of 86.727 small molecules for their ability to specifically reduce TASL-GFP fluorescence without affecting mCherry control signal (FIG. 2A-B). Reporter cells were incubated with compounds for 48 h before imaging as we aimed at monitoring degradation of the target protein, a process requiring time. mCherry signal was used as the basis for the detection of the cell area and images were evaluated for cell number and cell roundness to exclude highly toxic compounds and artefacts. Finally, the percentage of control (POC) normalized to cells treated with DMSO was evaluated with respect to GFP/mCherry ratio. Evaluation of the GFP signal was used as a filtering step to remove artefacts detected by the pipeline. 154 compounds that decreased the GFP/mCherry ratio by >15% and also reduced total GFP signal (>5% reduction) were identified as hits by the image analysis pipeline (FIG. 2C). After visual analysis 12 compounds were selected for validation in secondary assays (Table S1). First, their effect on endogenous TASL protein in TLR7/8-responsive human pDC cell line CAL-1 was monitored, and it was observed that the different compounds reduced TASL levels to various degrees already after 24 h. A salient feature of the SLC15A4-TASL axis is to specifically mediate IRF5 activation, while being dispensable for TLR7-9-induced NF-kB and MAPK activation. It was therefore decided to take advantage of this stringent criterion to identify those compounds that would specifically impair only IRF5. Developed was a robust microscopy-based assay to monitor nuclear translocation of IRF5 and NF-κB member p65/RelA upon stimulation with TLR7/8 agonist R848 in CAL-1. It was first confirmed that SLC15A4 and TASL knockout selectively affected IRF5 and not p65 translocation and 12 candidates were tested for their specificity. Five compounds displayed specific inhibition of IRF5 but left the NF-κB pathway unscathed (FIG. 2D). THP1 cells bearing an ISRE-driven reporter were used to investigate the kinetic profile as well as selectivity in inhibiting responses induced by agonists of endolysosomal versus plasma membrane TLRs.

Based on potency and kinetics, the compound C5 was selected for further characterization (FIG. 2E). C5 is an elongated molecule comprised of an aromatic 2-(4-ethoxyphenyl)-quinoline moiety followed by an amide bond and an aliphatic 2-methyl-1-propylpiperidine (FIG. 2F). To validate the scaffold and explore the chemical space at the aliphatic end of the compound, five analogues and an independently synthesized batch of C5 were obtained. All five analogues displayed a comparable level of activity in the THP1 ISRE reporter assay, highlighting that this activity is mediated by the common scaffold-see Example 4 for the detailed discussion of the structure activity relationships. Therefore the C5 compound was selected, which was named dTASL1 as its activity leads to TASL degradation, to assess effects on TLR7/8-induced signaling and responses. Key to any biological effect of dTASL1 would be its ability to modulate type I interferon (IFN) production. dTASL1 strongly suppressed R848-induced responses in CAL-1 pDCs, (FIG. 3E). Altogether these data demonstrate that dTASL1 specifically impairs TLR7/8-induced IRF5 activation and downstream type I IFN production in human monocytic and pDC cell lines.

### Example 2—Molecular Basis of Interference with a Highly Specific Protein Interaction Leading to Degradation of a Single Complex Component

Encouraged by the fact that the chemical entity of the present invention was showing the desired behavior and specificity profile, it became important to better clarify the molecular basis of this uncommon mechanism of action, i.e interference with a highly specific protein interaction leading to degradation of a single complex component. The effect of dTASL1 on complex assembly was first monitored. As expected, compound treatment reduced TASL-GFP levels in HEK293T cells co-expressing SLC15A4 (FIG. 3F). In contrast, dTASL-1 did not affect total levels of SLC15A4-GFP when expressed alone. When monitoring this effect by microscopy, a reduction of TASL-GFP levels accompanied by relocalisation was indeed observed (FIG. 3G). While total levels of SLC15A4-GFP were unaltered, localization appears to reflect some lysosomal rearrangement. To untangle the effects on TASL degradation from any possible lysosomal effects, advantage was taken of the ability of the present inventors to artificially localize the complex to the plasma membrane using ΔN-SLC15A4. dTASL1 did not affect levels or localization of ΔN-SLC15A4-GFP (FIG. 3F-G). Thus, in cells coexpressing ΔN-SLC15A4 and TASL-GFP, dTASL1 treatment severely reduced PM-associated TASL levels (FIG. 3G), strongly suggesting that dTASL1 is acting on the TASL-SLC15A4 complex directly, as it affects TASL levels also when the complex is experimentally localized at the plasma membrane. Altogether, these data exclude a direct role for the lysosome. To further rule out the residual possibility of an indirect effect, possibly related to TASL maturation or transport, dTASL1 competition assays with GFP-SLC15A4 associated with short N-terminal portions of TASL protein were performed. Co-expressed were GFP-SLC15A4 expressing cells with transiently transfected TASL-V5 fragments consisting of the first 94 and 193 amino acids, respectively. Immunofluorescence using V5 revealed the ability of the dTASL1 compound to reduce SLC15A4-localized V5 signal for both short TASL portions. These N-terminal fragments of TASL are predicted to be unstructured and unstable when not bound to SLC15A4. Thus, competition for SLC15A4 engagement of these protein portions by dTASL1 supports the notion that dTASL1 acts directly on SLC15A4 and competes for TASL binding, arguing against the involvement of other proteins, such as chaperone protein required for appropriate TASL maturation/folding.

As the data at this stage strongly points to SLC15A4 being the primary binding target of dTASL1, molecular docking to dTASL1 was performed. This simulation identified a cavity at the entrance of the channel as the most favorable dTASL1 position (FIG. 3J). In this tunnel, the aromatic moiety of dTASL1 appeared to be constrained, with all four lowest energy structures adopting almost identical conformations. The aliphatic moiety, on the other hand, was positioned above the channel. There was free space in this peptide “entrance” area and therefore the position of the 2-methyl-propylpiperidine moiety does not appear to be fixed. This is in good agreement with the limited effect of the chemical modifications introduced on the dTASL1 analogues, which were only altered in this unconstrained aliphatic region.

Given that all evidence obtained seems to confirm the mechanistic precision of this chemical agent, any further consideration of assessing the therapeutic potential required evidence for activity in human primary cells relevant for SLE pathophysiology. The effect of dTASL1 in human peripheral blood mononuclear cells (PBMCs) was assessed. In line with suppressed IRF5-dependent signaling, dTASL1 potently impaired TNF and to a lesser extent IL6 production upon R848 treatment of bulk PBMCs (FIG. 4A). It has been previously shown that the SLC15A4-TASL module was required for TNF production in primary human monocytes. Hence, the effect of dTASL1 on purified CD14⁺ cells was tested, and a significant reduction in this proinflammatory cytokine was observed (FIG. 4B). Among endolysosomal TLR responsive cells, B cells play a central role in SLE pathogenesis and are the targets of current therapeutics. The impact of dTASL1 on TLR7/8 responses by directly monitoring IRF5 by confocal microscopy in B cells was assessed. Remarkably, dTASL1 significantly reduced IRF5 nuclear translocation, without affecting the activation of NF-κB p65 (FIG. 4C-D). These data demonstrate that dTASL1 activity extends to disease-relevant human primary immune cells and highlights that the exquisite selectivity for targeting the IRF5 signaling branch was also preserved in primary human B cells.

These effects were also confirmed with cells derived from SLE patients (FIG. 4E-F).

### Example 3—SAR Studies of the Compound of Formula (I)

The following compounds were assayed for their activity in the degradation assay (THP1 DUAL/ISRE cell reporter assay), as described herein:

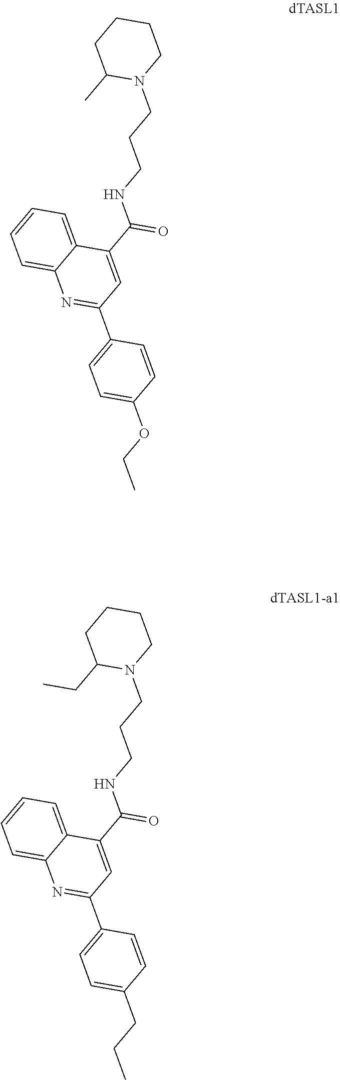 [SMILES: CCOc1ccc(-c2cc(C(=O)NCCCN3CCCCC3C)c3ccccc3n2)cc1 ; CCCc1ccc(-c2cc(C(=O)NCCCN3CCCCC3CC)c3ccccc3n2)cc1]

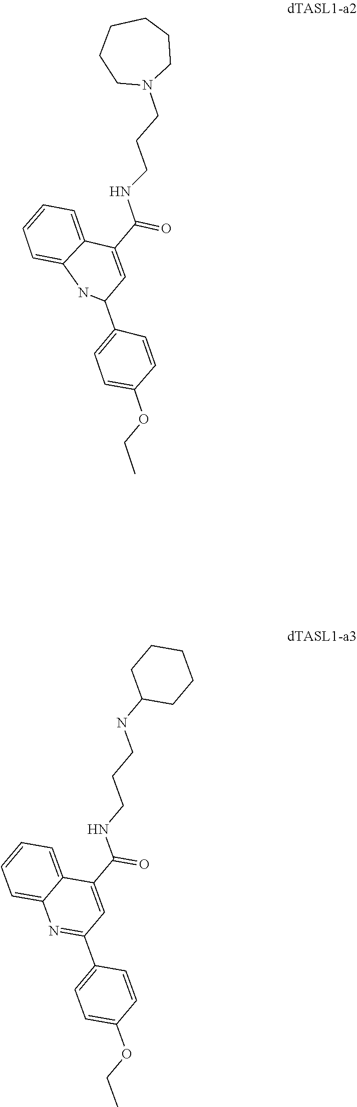 [SMILES: CCOc1ccc(C2C=C(C(=O)NCCCN3CCCCCC3)c3ccccc3N2)cc1 ; CCOc1ccc(-c2cc(C(=O)NCCCNC3CCCCC3)c3ccccc3n2)cc1]

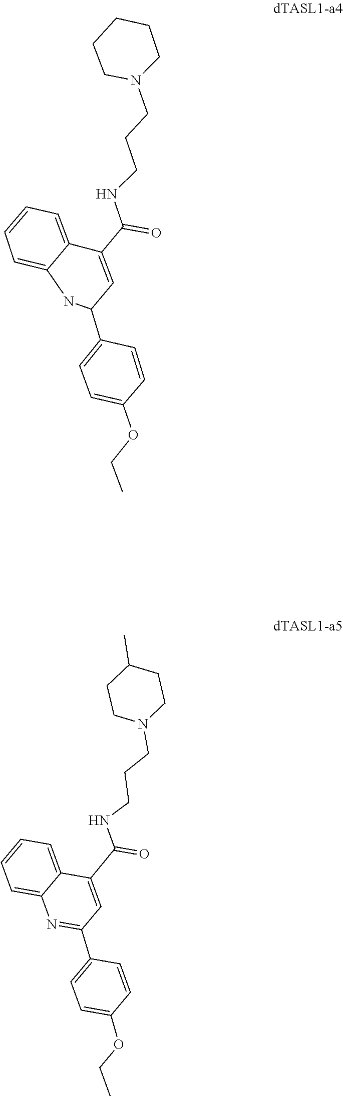 [SMILES: CCOc1ccc(C2C=C(C(=O)NCCCN3CCCCC3)c3ccccc3N2)cc1 ; CCOc1ccc(-c2cc(CC(=O)NCCCN3CCC(C)CC3)c3ccccc3n2)cc1]

The results are summarized in the following Table 2:

TABLE 2
Compound IC50s in THP1 dual cell assay after 24 h and
48 h treatment before R848 stimulation, respectively.

| | IC50 24 h | SEM 24 h | IC50 48 h | SEM 48 h | |
|---|---|---|---|---|---|
| Compound | [μM] | [μM] | [μM] | [μM] | Vendor |
| dTASL1 | 2.230 | 0.206 | 0.933 | 0.455 | ChemDiv |
| dTASL1-a1 | 2.055 | 0.156 | 1.225 | 1.142 | ChemDiv |
| dTASL1-a2 | 3.494 | 0.463 | 2.965 | 1.570 | ChemDiv |
| dTASL1-a3 | 8.247 | 1.384 | 1.494 | 0.857 | ChemDiv |
| dTASL1-a4 | 4.155 | 0.507 | 2.395 | 1.510 | ChemDiv |
| dTASL1-a5 | 8.902 | 5.742 | 2.801 | 1.708 | ChemDiv |

The data are shown in FIGS. 5 and 6.

### Example 4-Synthesis of dTASL1

### 2-(4-ethoxyphenyl)-N-(3-(2-methylpiperidin-1-yl) propyl) quinoline-4-carboxamide

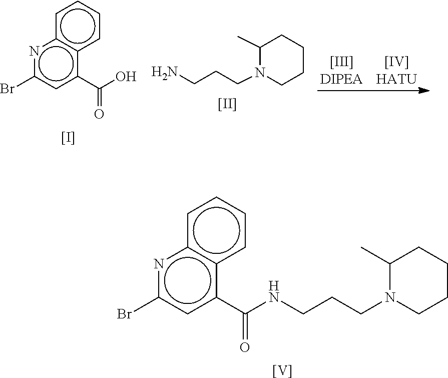

A solution of 2-bromoquinoline-4-carboxylic acid (1 g, 1 Eq, 4 mmol), 2-bromoquinoline-4-carboxylic acid (1 g, 1 Eq, 4 mmol), 2-(3H-[1,2,3]triazolo[4,5-b]pyridin-3-yl)-1,1,3,3-tetramethylisouronium hexafluorophosphate (V) (3 g, 2 Eq, 8 mmol), and N-ethyl-N-isopropylpropan-2-amine (2 g, 4 Eq, 0.02 mol) in dry DMF (25 mL) was stirred for 12 hour at 20 °C. After removal of the solvent by evaporation, the crude was purified by flash column chromatography (DCM-MEOH gradient) to give 2-bromo-N-(3-(2-methylpiperidin-1-yl) propyl) quinoline-4-carboxamide. Yield 65%.

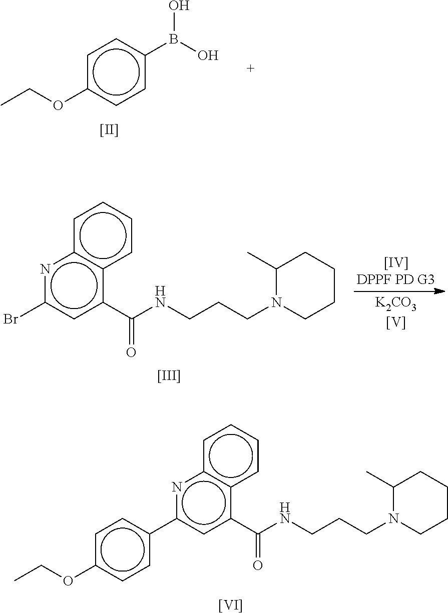

(4-ethoxyphenyl) boronic acid (0.5 g, 1.2 Eq, 3 mmol), 2-bromo-N-(3-(2-methylpiperidin-1-yl) propyl) quinoline-4-carboxamide (1 g, 1 Eq, 3 mmol) were weighed into a 50 mL Schlenk tube. Than DPPF Pd G3 (0.02 g, 0.01 Eq, 0.03 mmol) and potassium carbonate (0.7 g, 2 Eq, 5 mmol) were added. The powder mixture was suspended in 1,4-Dioxane (10 mL) and H2O (2.5 mL) and degassed with bubbling through argon. The reaction mixture was heated to 90 °C for 18 hour. The black reaction mixture was diluted with 50 mL ethyl acetate and washed with water (2*50 mL), brine (1*50 mL), and the organic phase was concentrated under vacuum. The crude was dissolved in ~1 mL dichloromethane an loaded onto a silica column, and purified via column chromatography (gradient DCM/MEOH 0 to 95) to afford 2-(4-ethoxyphenyl)-N-(3-(2-methylpiperidin-1-yl) propyl) quinoline-4-carboxamide. Yield 69%.

1. A compound of formula (I): 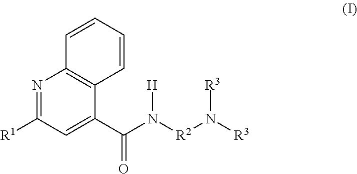
or a pharmaceutically acceptable salt thereof, wherein:
R¹ is aryl or heteroaryl, optionally substituted with one or more R^{S2},
R² is C₁₋₅ alkylene, optionally substituted with one or more R^{S1},
each R³ is independently selected from C₁₋₅ alkyl, C₂₋₅ alkenyl, C₂₋₅ alkynyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said —(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2}, or
both R³ are joined together to form together with the N atom that otherwise carries both R³ a heterocyclic ring, optionally substituted with one or more R^{S2}, or —R²—NR³R³ are taken together to form: 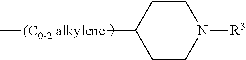
wherein R³ is selected from C₁₋₅ alkyl, C₂₋₅ alkenyl, C₂₋₅ alkynyl, (C₀₋₃ alkylene)-cycloalkyl, (C₀₋₃ alkylene)-heterocycloalkyl, (C₀₋₃ alkylene)-aryl and (C₀₋₃ alkylene)-heteroaryl, wherein said alkyl, said alkenyl, said alkynyl and the alkylene moiety in said-(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl are each optionally substituted with one or more R^{S1}, wherein the cycloalkyl moiety in said —(C₀₋₃ alkylene)-cycloalkyl, the heterocycloalkyl moiety in said-(C₀₋₃ alkylene)-heterocycloalkyl, the aryl moiety in said-(C₀₋₃ alkylene)-aryl and the heteroaryl moiety in said-(C₀₋₃ alkylene)-heteroaryl are each optionally substituted with one or more R^{S2};
wherein each R^{S1} is independently selected from —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)—COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl); and
wherein each R^{S2} is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —(C₁₋₆ alkylene)-OH, —(C₁₋₆ alkylene)-O(C₁₋₆ alkyl), —SH, —S(C₁₋₆ alkyl), —S(C₁₋₆ alkylene)-SH, —S(C₁₋₆ alkylene)-S(C₁₋₆ alkyl), —(C₁₋₆ alkylene)-SH, —(C₁₋₆ alkylene)-S(C₁₋₆ alkyl), —NH₂, —NH(C₁₋₆ alkyl), —N(C₁₋₆ alkyl) (C₁₋₆ alkyl), —NH—OH, —N(C₁₋₆ alkyl)-OH, —NH—O(C₁₋₆ alkyl), —N(C₁₋₆ alkyl)-O(C₁₋₆ alkyl), -HaI, C₁₋₆ haloalkyl, —O—(C₁₋₆ haloalkyl), —CN, —NO₂, —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), —SO—(C₁₋₆ alkyl), —(C₀₋₄ alkylene)-carbocyclyl, and —(C₀₋₄ alkylene)-heterocyclyl, wherein the carbocyclyl group in said-(C₀₋₄ alkylene)-carbocyclyl and the heterocyclyl group in said-(C₀₋₄ alkylene)-heterocyclyl are each optionally substituted with one or more groups independently selected from C₁₋₆ alkyl, C₂₋₆ alkenyl, C₂₋₆ alkynyl, halogen, C₁₋₆ haloalkyl, —O—(C₁₋₆ haloalkyl), —CN, —OH, —O(C₁₋₆ alkyl), —SH, —S(C₁₋₆ alkyl), —NH₂, —NH(C₁₋₆ alkyl), —N(C₁₋₆ alkyl) (C₁₋₆ alkyl), —CHO, —CO(C₁₋₆ alkyl), —COOH, —COO(C₁₋₆ alkyl), —O—CO(C₁₋₆ alkyl), —CO—NH₂, —CO—NH(C₁₋₆ alkyl), —CO—N(C₁₋₆ alkyl) (C₁₋₆ alkyl), —NH—CO(C₁₋₆ alkyl), —N(C₁₋₆ alkyl)-CO(C₁₋₆ alkyl), —NH—COO(C₁₋₆ alkyl), —N(C₁₋₆ alkyl)-COO(C₁₋₆ alkyl), —O—CO—NH(C₁₋₆ alkyl), —O—CO—N(C₁₋₆ alkyl) (C₁₋₆ alkyl), —SO₂—NH₂, —SO₂—NH(C₁₋₆ alkyl), —SO₂—N(C₁₋₆ alkyl) (C₁₋₆ alkyl), —NH—SO₂—(C₁₋₆ alkyl), —N(C₁₋₆ alkyl)-SO₂—(C₁₋₆ alkyl), —SO₂—(C₁₋₆ alkyl), and —SO—(C₁₋₆ alkyl).

2. The compound of claim 1, wherein R¹ is phenyl substituted with one or more R^{S2}.

3. The compound of claim 1 or 2, wherein R¹ is phenyl substituted with —O(C₁₋₆ alkyl).

4. The compound of any one of claims 1 to 3, wherein R¹ is 4-ethoxyphenyl.

5. The compound of any one of claims 1 to 4, wherein R² is C₂₋₄ alkylene.

6. The compound of claim 5, wherein R² is selected from —CH₂CH₂—, —CH₂CH₂CH₂—, —CH₂CH₂CH₂CH₂—, and —CH₂CH(CH₃)CH₂—.

7. The compound of claim 5 or 6, wherein R² is-CH₂CH₂CH₂—.

8. The compound of any one of claims 1 to 7, wherein each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said —(C₀₋₃ alkylene)-aryl are each optionally substituted with one or more R^{S2}.

9. The compound of claim 8, wherein each R³ is independently selected from methyl, ethyl, propyl, isopropyl, butyl, isobutyl, cyclohexyl and benzyl.

10. The compound of claim 8 or 9, with the proviso that not both R³ are unsubstituted methyl and with the proviso that not both R³ are unsubstituted ethyl.

11. The compound of any one of claims 1 to 7, wherein both R³ are joined together to form together with the N atom that otherwise carries both R³ a heterocyclic ring optionally substituted with one or more R^{S2}.

12. The compound of any one of claim 1 to 7 or 11, wherein both R³ are joined together to form together with the N atom that otherwise carries both R³ a heterocycloalkyl ring optionally substituted with one or more R^{S2}.

13. The compound of claim 11, wherein the heterocyclic ring optionally substituted with one or more R^{S2} is a piperidine ring or pyrrolidine ring, optionally substituted with one or more R^{S2}, preferably a 2-methylpiperidine ring.

14. The compound of any one of claims 1 to 7, wherein —R²—NR³R³ are taken together to form: 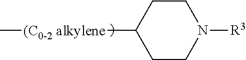
preferably wherein R³ is selected from methyl, ethyl, propyl, isopropyl, butyl, isobutyl, cyclohexyl and benzyl.

15. The compound of claim 1, wherein
R¹ is 4-ethoxyphenyl;
R² is C₂₋₄ alkylene; and
both R³ are joined together to form together with the N atom that otherwise carries both R³ a heterocycloalkyl ring optionally substituted with one or more R^{S2}.

16. The compound of claim 1, wherein
R¹ is 4-ethoxyphenyl;
R² is C₂₋₄ alkylene; and
both R³ are joined together to form together with the N atom that otherwise carries both R³ a heterocyclic ring optionally substituted with one or more C₁₋₆ alkyl groups.

17. The compound of claim 1, wherein
R¹ is 4-ethoxyphenyl;
R² is C₂₋₄ alkylene; and
each R³ is independently selected from C₁₋₅ alkyl, —(C₀₋₃ alkylene)-cycloalkyl and —(C₀₋₃ alkylene)-aryl, wherein said alkyl, and the alkylene moiety in said-(C₀₋₃ alkylene)-cycloalkyl and —(C₀₋₃ alkylene)-aryl are each optionally substituted with one or more R^{S1}, wherein the cycloalkyl moiety in said-(C₀₋₃ alkylene)-cycloalkyl and the aryl moiety in said-(C₀₋₃ alkylene)-aryl are each optionally substituted with one or more R^{S2}.

18. The compound of claim 17, with the proviso that not both R³ are unsubstituted methyl and with the proviso that not both R³ are unsubstituted ethyl.

19. The compound of any one of claims 15 to 18, wherein R² is C₃₋₄ alkylene.

20. The compound of any one of claims 1 to 19, wherein the following compounds: 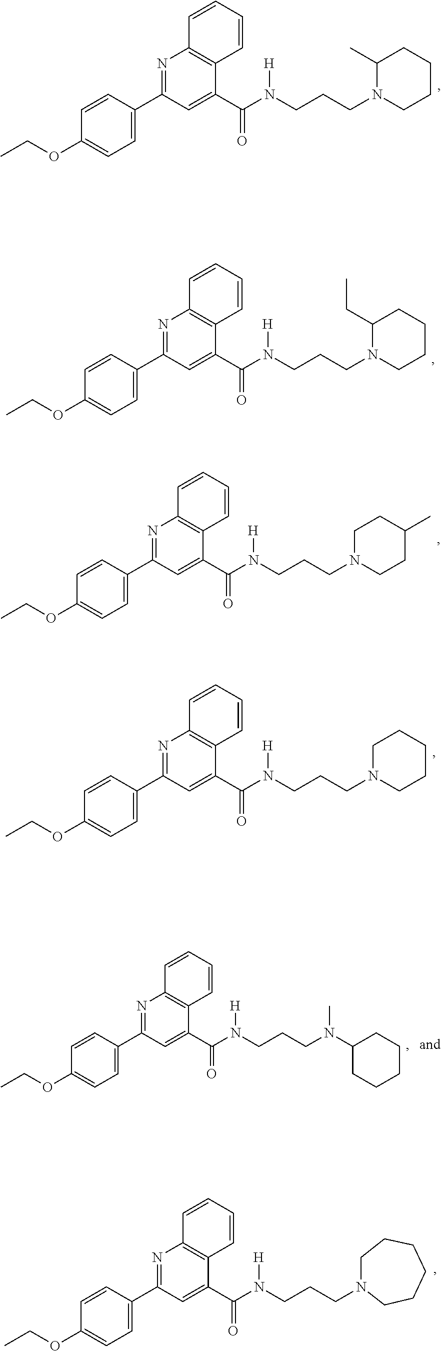
are excluded from formula (I).

21. The compound of claim 1, wherein the compound is a compound selected from the following compounds or their pharmaceutically acceptable salts:

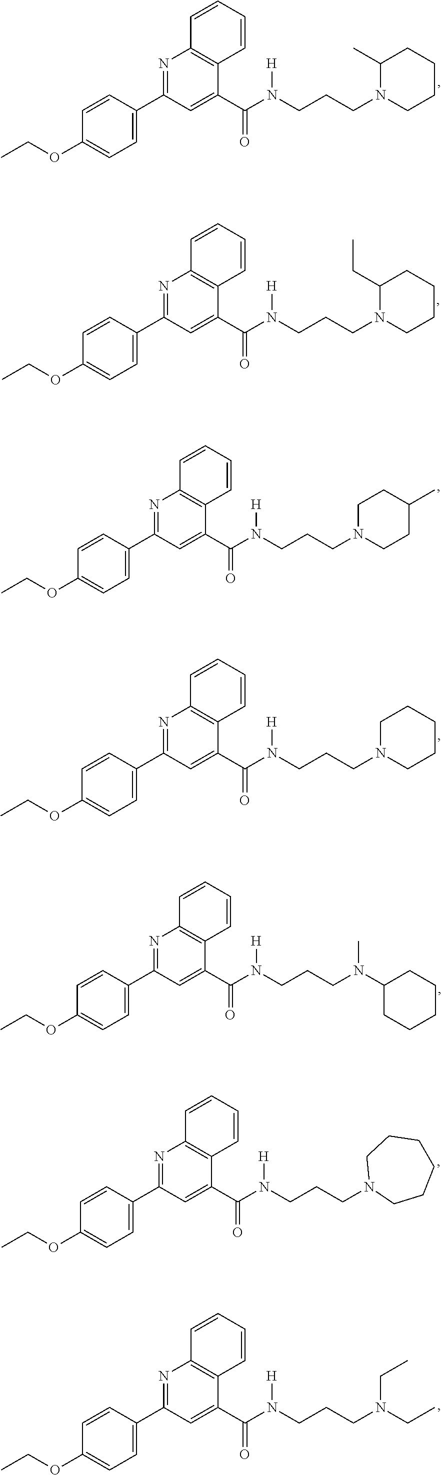

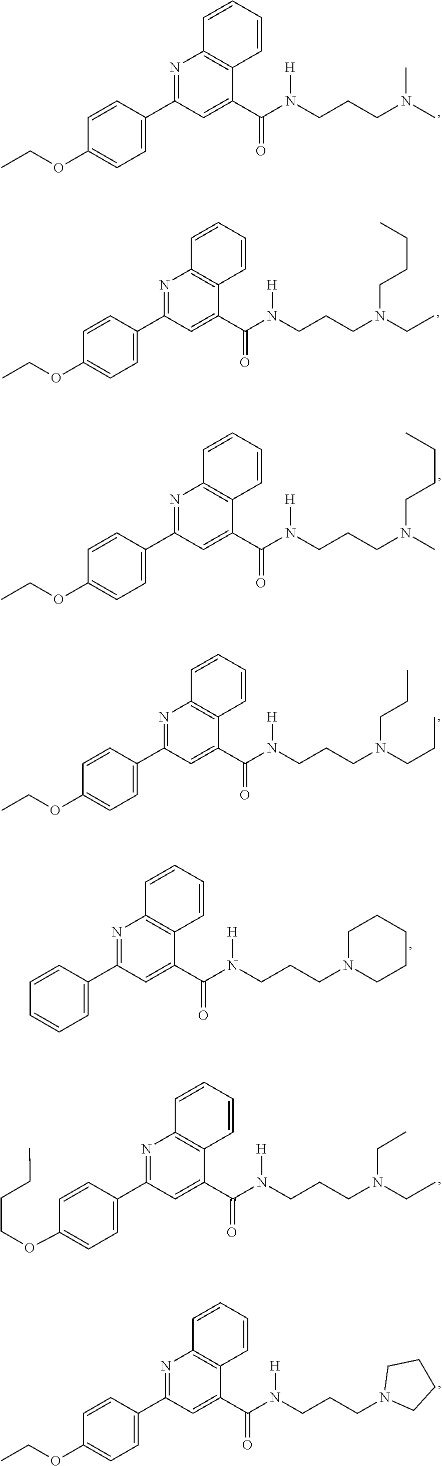

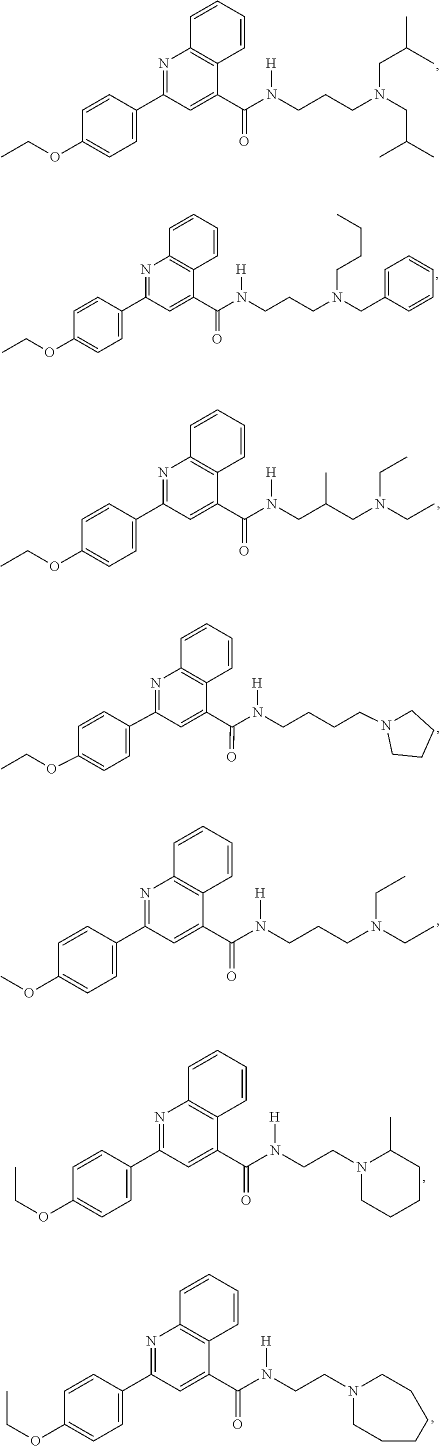

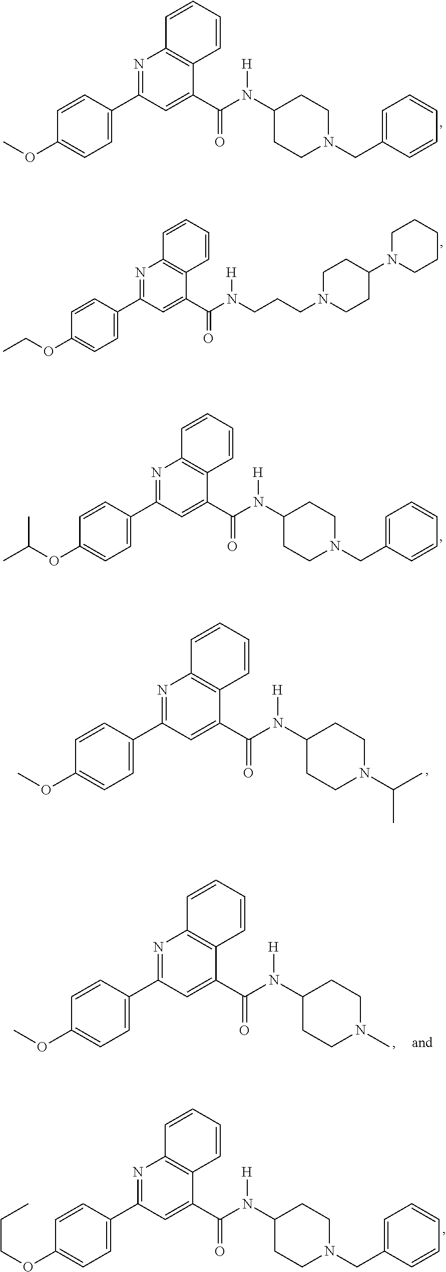

22. A pharmaceutical composition comprising the compound of any one of claims 1 to 21 and at least one pharmaceutically acceptable carrier.

23. The compound of any one of claims 1 to 21 or the pharmaceutical composition of claim 22 for use as a medicament.

24. The compound of any one of claims 1 to 21 or the pharmaceutical composition of claim 22 for use in treatment or prevention of an autoimmune disorder or inflammatory condition.

25. The compound for use or the pharmaceutical composition for use of claim 24, for use in the treatment or prevention of an autoimmune disorder, wherein the autoimmune disorder is selected from systemic lupus erythematosus, rheumatoid arthritis, scleroderma, Sjögren's syndrome, polymyositis/dermatomyositis, primary biliary cirrhosis, multiple sclerosis, oral ulcers, periodontitis, Behçet's disease, myasthenia gravis, and ankylosing spondylitis.

26. The compound for use or the pharmaceutical composition for use of claim 25,
wherein the autoimmune disorder is systemic lupus erythematosus.

27. The compound for use or the pharmaceutical composition for use of claim 24, for use in the treatment or prevention of an inflammatory condition,
wherein the inflammatory condition is selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis, preferably wherein the inflammatory condition is selected from inflammatory bowel disease, psoriasiform dermatitis, and endosomal TLR-dependent inflammation.

28. The compound for use or the pharmaceutical composition for use of any one of claims 24 to 27, wherein the autoimmune disorder is a disorder associated with SLC15 peptide transporter, and/or wherein said compound or said pharmaceutical composition inhibits SLC15 peptide transporter.

29. The compound for use of any one of claims 24 to 28, wherein the compound is the compound according to claim 16.


SEQUENCE LISTING (18 sequences)

SEQ ID NO: 1 - DNA, 38 bp, synthetic construct
  source 1..38: mol_type = other DNA
ccatgctgagcgagggctatctgagcggactggagtat

SEQ ID NO: 2 - DNA, 38 bp, synthetic construct
  source 1..38: mol_type = other DNA
cagcatactccagtccgctcagatagccctcgctcagc

SEQ ID NO: 3 - DNA, 41 bp, synthetic construct
  source 1..41: mol_type = other DNA
ccatgctgagcgagggctatctgagcggactggagtattgg

SEQ ID NO: 4 - DNA, 41 bp, synthetic construct
  source 1..41: mol_type = other DNA
cagcccaatactccagtccgctcagatagccctcgctcagc

SEQ ID NO: 5 - DNA, 44 bp, synthetic construct
  source 1..44: mol_type = other DNA
ccatgctgagcgagggctatctgagcggactggagtattggaac

SEQ ID NO: 6 - DNA, 44 bp, synthetic construct
  source 1..44: mol_type = other DNA
cagcgttccaatactccagtccgctcagatagccctcgctcagc

SEQ ID NO: 7 - DNA, 47 bp, synthetic construct
  source 1..47: mol_type = other DNA
ccatgctgagcgagggctatctgagcggactggagtattggaacgac

SEQ ID NO: 8 - DNA, 47 bp, synthetic construct
  source 1..47: mol_type = other DNA
cagcgtcgttccaatactccagtccgctcagatagccctcgctcagc

SEQ ID NO: 9 - RNA, 21 nt, synthetic construct
  source 1..21: mol_type = other RNA
gggcagccttctacggaattt

SEQ ID NO: 10 - RNA, 21 nt, synthetic construct
  source 1..21: mol_type = other RNA
caaccagaccatcggcaattt

SEQ ID NO: 11 - DNA, 25 bp, synthetic construct
  source 1..25: mol_type = other DNA
caccgggagcgatcctgtcgttagg

SEQ ID NO: 12 - DNA, 25 bp, synthetic construct
  source 1..25: mol_type = other DNA
aaaccctaacgacaggatcgctccc

SEQ ID NO: 13 - DNA, 25 bp, synthetic construct
  source 1..25: mol_type = other DNA
caccgtattacaaccactcctcaca

SEQ ID NO: 14 - DNA, 25 bp, synthetic construct
  source 1..25: mol_type = other DNA
aaactgtgaggagtggttgtaatac

SEQ ID NO: 15 - DNA, 25 bp, synthetic construct
  source 1..25: mol_type = other DNA
caccggtagaaatggaatcctccat

SEQ ID NO: 16 - DNA, 25 bp, synthetic construct
  source 1..25: mol_type = other DNA
aaacatggaggattccatttctacc

SEQ ID NO: 17 - DNA, 25 bp, synthetic construct
  source 1..25: mol_type = other DNA
caccgctgaattaatggccatcacc

SEQ ID NO: 18 - DNA, 25 bp, synthetic construct
  source 1..25: mol_type = other DNA
aaacggtgatggccattaattcagc